(19) United States
(12) Patent Application Publication
(10) Pub. No.: US 2026/0144901 A1
(43) Pub. Date: May 28, 2026

(54) CUSTOMIZABLE LYOPHILIZED AGENT FOR IMAGE-GUIDED RADIOTHERAPY AND DRUG DELIVERY

(71) Applicant: THE JOHNS HOPKINS UNIVERSITY, Baltimore, MD (US)

(72) Inventors: Wilfred Ngwa, Baltimore, MD (US); Michele Moreau, Baltimore, MD (US)

(21) Appl. No.: 19/395,539

(22) Filed: Nov. 20, 2025

Related U.S. Application Data
(60) Provisional application No. 63/724,181, filed on Nov. 22, 2024.

(51) Int. Cl. A61K 49/18; A61K 9/10; A61K 9/19; A61K 31/352; A61K 39/395; A61K 49/04; A61K 49/12

(52) U.S. Cl. CPC A61K 49/1887; A61K 9/10; A61K 9/19; A61K 31/352; A61K 39/3955; A61K 49/0423; A61K 49/126

(57) ABSTRACT

The present disclosure provides a customizable lyophilized agent and its use for cancer treatment. The agent comprises a biocompatible lyophilized matrix and nanoparticles incorporated within the lyophilized matrix. The lyophilized matrix is reconstitutable with a biocompatible solvent to form a colloid. The nanoparticles provide imaging contrast for at least one imaging modality. The nanoparticles may provide contrast for both computed tomography and magnetic resonance imaging. The lyophilized matrix may be reconstituted with an aqueous solution of a therapeutic agent, allowing for customization of the agent for specific treatment needs.

### CROSS-REFERENCE TO RELATED APPLICATIONS

This application claims the benefit of priority under 35 U.S.C. § 119 (e) of U.S. Provisional Application No. 63/724,181 filed Nov. 22, 2024. The disclosure of the prior application is considered part of and is herein incorporated by reference in the disclosure of this application in its entirety.

### STATEMENT REGARDING FEDERALLY SPONSORED RESEARCH OR DEVELOPMENT

This invention was made with government support under grant CA239042 awarded by the National Institutes of Health. The government has certain rights in the invention.

### TECHNICAL FIELD

The present disclosure generally relates to customizable lyophilized agents, methods of use and methods for preparing said lyophilized agents.

### BACKGROUND

Image-guided radiation therapy (IGRT) is a widely used technique in cancer treatment that employs medical imaging to improve the precision and accuracy of radiation delivery to tumors. IGRT typically utilizes fiducial markers to aid in aligning the tumor target area for accurate beam delivery. These fiducial markers provide a reference point that can be visualized on imaging scans to ensure proper positioning of the radiation beams.

Conventional fiducial markers used in IGRT include solid markers made of materials such as gold or titanium, as well as liquid fiducial markers. While these traditional markers serve the purpose of providing imaging contrast, they are limited in their functionality. Solid markers may migrate within tissue, require specific needle sizes for implantation, and can potentially produce imaging artifacts or perturb the radiation dose distribution. Liquid fiducials, while addressing some of these issues, may have limitations related to storage conditions and lack additional therapeutic capabilities.

There is a growing interest in developing multifunctional biomaterials that can serve not only as fiducial markers but also as drug delivery systems to enhance therapeutic outcomes. Such smart radiotherapy biomaterials (SRBs) have the potential to combine imaging capabilities with localized drug delivery, potentially improving treatment efficacy and patient outcomes.

However, the development of SRBs that are stable, easy to use, and suitable for various clinical settings, including low-resource environments, remains challenging. Issues such as cold chain logistics, long-term stability, and the ability to customize the therapeutic payload at the point of care are important considerations in the design of next-generation fiducial markers.

Additionally, there is a need for fiducial markers that can provide contrast across multiple imaging modalities, such as computed tomography (CT) and magnetic resonance imaging (MRI), to enhance versatility in different clinical scenarios. The ability to track these markers over extended periods and their potential to address tumor microenvironment factors, such as hypoxia, are also areas of interest in improving radiotherapy outcomes.

Pancreatic cancer is a deadly disease accounting for approximately 67,000 new cases a year with a less than 10% 5-year survival rate. This low 5-year survival rate is pronounced because at the point of diagnosis, 50-60% of patients are distant metastatic and 25-30% with regional disease. Current treatments include surgery, chemotherapy, radiation therapy, targeted therapy and immunotherapy. However, surgery is typically only available for patients who are diagnosed in the early stages with tumors that have clear margins for resection. Therefore chemotherapy, radiation therapy, targeted therapy and immunotherapy remain the most common forms of treatment; but pancreatic cancer is notoriously resistant to these treatments due to its immunosuppressive tumor microenvironment.

Immunoadjuvants are therefore used stand alone or in combination with other therapies to enhance the immune system response thereby increasing efficacy. Flavonoids are an example of an immunoadjuvant naturally derived from plants and fruits and have shown anti-inflammatory and anti-cancer properties. With the ability to engage pathways in both the innate and adaptive immune system, making them great immunoadjuvants. In addition, they have been shown to be an effective anti-cancer drug due to their involvement in inhibiting cancer pathways, one important pathway flavonoids have shown involvement in is controlling and inhibiting the NF-κβ pathway which is associated with inflammation, immunosuppression and treatment resistance. Previous studies have shown the use of flavonoids in enhancing treatment efficacy in cancer, including colorectal cancer, breast cancer, and glioma.

Immunoadjuvants, however, are limited by systemic toxicities, especially when combined with another form of therapy such as radiation therapy. To overcome this obstacle, smart radiotherapy biomaterials can be used in treatment to directly administer the drug into the tumor over time. Smart biomaterials are a significant drug delivery system because they are programmed to react to their environment and stimuli. This allows for greater control in completing their intended effect while ideally reducing off target effects. One example of smart biomaterials in cancer is a thermosensitive smart hydrogel which can activate the Kv11.1 K⁺ channel which can inhibit tumor growth. In the context of radiation therapy, smart biomaterials can meet two needs in the clinic. They can be formulated to act as a fiducial marker in radiation therapy which is crucial in tracking tumor movement over the course of therapy cost. Additionally, as well as be loaded with immunoadjuvant drugs which can improve the therapeutic index by sensitizing the immune system.

### SUMMARY OF THE INVENTION

The present disclosure is based on the seminal discovery of an immunotherapeutic potential of CLARITY Biomaterial loaded caflanone. A significant decrease in pancreatic cancer cell proliferation was observed for cells that received both Caflanone combined with radiation therapy.

In one embodiment, the present disclosure provides a customizable lyophilized agent, comprising: a biocompatible lyophilized matrix; and nanoparticles incorporated within the lyophilized matrix, wherein the lyophilized matrix is reconstitutable with a biocompatible solvent to form a colloid, and wherein the nanoparticles provide imaging contrast for at least one imaging modality.

In some aspects, the nanoparticles comprise at least one of manganese oxide nanoparticles, cerium oxide nanoparticles, gold nanoparticles, titanium dioxide nanoparticles, or gadodiamide nanoparticles.

In some aspects, the nanoparticles provide imaging contrast for computed tomography (CT), magnetic resonance imaging (MRI), or a combination thereof.

In some aspects, the biocompatible lyophilized matrix comprises chitosan and sodium alginate.

In some aspects, the customizable lyophilized agent further comprises a therapeutic agent incorporated within the lyophilized matrix. In some aspects, the therapeutic agent is an antibody, an immunoadjuvant, a small molecule drug, a nucleic acid, a peptide, or a combination thereof. In some aspects, therapeutic agent is an immunoadjuvant. In some aspects, the antibody is an anti-CD40 monoclonal antibody. In some aspects, the small molecule drug is caflanone.

In some aspects, the lyophilized matrix is reconstitutable with an aqueous solution of the therapeutic agent.

In one embodiment, the present disclosure provides a method of imaging a subject using a customizable lyophilized agent, comprising: reconstituting a lyophilized agent with a biocompatible solvent to form a colloid, wherein the lyophilized agent comprises a biocompatible lyophilized matrix and nanoparticles incorporated within the lyophilized matrix; administering the colloid to the subject; and imaging the subject using at least one imaging modality, wherein the nanoparticles provide imaging contrast for the at least one imaging modality.

In some aspects, the at least one imaging modality comprises computed tomography (CT), magnetic resonance imaging (MRI), or a combination thereof.

In some aspects, the nanoparticles comprise at least one of manganese oxide nanoparticles, cerium oxide nanoparticles, gold nanoparticles, titanium dioxide nanoparticles, or gadodiamide nanoparticles.

In some aspects, the method further comprising administering radiotherapy to the subject based on imaging data obtained from the at least one imaging modality.

In some aspects, the biocompatible solvent comprises an aqueous solution of a therapeutic agent. In some aspects, the therapeutic agent is an antibody, an immunoadjuvant, a small molecule drug, a nucleic acid, a peptide, or a combination thereof. In some aspects, the therapeutic agent is an immunoadjuvant. In some aspects, the antibody is an anti-CD40 monoclonal antibody. In some aspects, the small molecule drug is caflanone.

In one embodiment, the present disclosure provides a method of treatment using image-guided radiation therapy (IGRT) comprising administering to the subject the customizable lyophilized agent described above.

In some aspects, the treatment is for cancer. In some aspects, the cancer is arteriovenous malformations, bone cancer, brain cancer, breast cancer, chondrosarcoma, chordoma, colon cancer, esophageal cancer, Ewing's sarcoma, head and neck cancers, leukemia, liver cancer, lung cancer, lymphoma, metastatic brain cancer, meningioma, neuroma, non-Hodgkin's lymphoma, osteosarcoma, pancreatic cancer, paranasal sinus cancer, prostate cancer, soft tissue sarcomas, spinal cord tumors, trigeminal neuralgia, or a combination thereof. In some aspects, the cancer is pancreatic cancer.

In some aspects, the method further comprising administering a therapeutic agent. In some aspects, the therapeutic agent is an antibody, an immunoadjuvant, a small molecule drug, a nucleic acid, a peptide, or a combination thereof. In some aspects, the therapeutic agent is an immunoadjuvant. In some aspects, the antibody is an anti-CD40 monoclonal antibody. In some aspects, the small molecule drug is caflanone.

In some aspects, the biocompatible solvent comprises an aqueous solution of a therapeutic agent.

In one embodiment, the present disclosure provides a method of preparing a customizable lyophilized agent, comprising: mixing a biocompatible hydrogel with nanoparticles to form a mixture; freezing the mixture; and lyophilizing the frozen mixture to produce a lyophilized agent, wherein the lyophilized agent is reconstitutable with a biocompatible solvent to form a colloid, and wherein the nanoparticles provide imaging contrast for at least one imaging modality.

In some aspects, the biocompatible hydrogel comprises chitosan and sodium alginate.

In some aspects, the nanoparticles comprise at least one of manganese oxide nanoparticles, cerium oxide nanoparticles, gold nanoparticles, titanium dioxide nanoparticles, or gadodiamide nanoparticles.

In some aspects, the nanoparticles provide imaging contrast for both computed tomography (CT), magnetic resonance imaging (MRI), or a combination thereof.

In some aspects, the method further comprising incorporating a therapeutic agent within the mixture prior to freezing.

In some aspects, the therapeutic agent is an antibody, an immunoadjuvant, a small molecule drug, a nucleic acid, a peptide, or a combination thereof. In some aspects, the therapeutic agent is an immunoadjuvant. In some aspects, the antibody is an anti-CD40 monoclonal antibody. In some aspects, the small molecule drug is caflanone.

In some aspects, the method further comprising reconstituting the lyophilized agent with an aqueous solution of the therapeutic agent.

In some aspects, the biocompatible solvent comprises an aqueous solution of a therapeutic agent.

### BRIEF DESCRIPTION OF DRAWINGS

Non-limiting and non-exhaustive examples are described with reference to the following figures.

FIG. 1 illustrates the evolution of smart radiotherapy biomaterials.

FIG. 2 illustrates a process for biomaterial production.

FIGS. 3A-3B illustrate close-up views of containers holding different biomaterial formulations, according to aspects of the present disclosure. FIG. 3A illustrates a lyophilized agent formulated with gold nanoparticles (GNP). FIG. 3B illustrates a lyophilized agent formulated with manganese oxide nanoparticles (Mn₂O₃).

FIGS. 4A-4C illustrate transmission electron microscopy images of biomaterial formulations, according to an embodiment. FIG. 4A illustrates a TEM image of the lyophilized agent formulated with manganese oxide (Mn₂O₃) nanoparticles. FIG. 4B illustrates a TEM image of the lyophilized agent (Mn₂O₃) loaded with anti-CD40. FIG. 4C illustrates a TEM image of the lyophilized agent (Mn₂O₃) loaded with caflanone.

FIGS. 5A-5B illustrate size distribution graphs for a biomaterial sample, according to aspects of the present disclosure. FIG. 5A illustrates a particle concentration distribution graph for the lyophilized agent. FIG. 5B illustrates a volume distribution graph for the same lyophilized agent.

FIG. 6A-6B depict size distribution graphs for another biomaterial sample, according to an embodiment. FIG. 6A illustrates a particle concentration distribution graph for the lyophilized agent. FIG. 6B illustrates a volume distribution graph for the same lyophilized agent.

FIG. 7 shows MRI images of mice injected with a biomaterial over time, according to aspects of the present disclosure.

FIG. 8 shows MRI images of mice treated with a different biomaterial formulation over time, according to an embodiment.

FIG. 9 shows CT images of mice injected with a biomaterial over time, according to aspects of the present disclosure.

FIG. 10 shows CT images of mice treated with a different biomaterial formulation over time, according to an embodiment.

FIG. 11 illustrates medical imaging views of a kidney before and after biomaterial injection, according to aspects of the present disclosure.

FIG. 12 illustrates medical imaging scans of a breast area before and after biomaterial injection, according to an embodiment.

FIGS. 13A-13B depict graphs showing biomaterial efficacy in pancreatic cancer treatment, according to aspects of the present disclosure. FIG. 13A illustrates a graph showing the changes in pancreatic tumor volume over time for different treatment groups. FIG. 13B illustrates a survival curve for the same treatment groups over the same period.

FIGS. 14A-14B illustrate transmission electron microscopy images of a biomaterial in different forms, according to an embodiment. FIG. 14A illustrates the TEM image of the lyophilized agent in powder form reveals a distribution of spherical nanoparticles with varying sizes, dispersed throughout a less dense background matrix. FIG. 14B illustrates the TEM image of the lyophilized agent suspended in PBS shows a more complex structure, with numerous particles of varying sizes dispersed throughout the field of view.

FIGS. 15A-15C illustrate transmission electron microscopy images of biomaterial formulations, according to aspects of the present disclosure. FIG. 15A illustrates the TEM image of the lyophilized agent in powder form reveals a distribution of spherical nanoparticles with varying sizes, dispersed throughout a less dense background matrix. FIG. 15B illustrates the TEM image of the lyophilized agent suspended in PBS shows a more complex structure, with numerous particles of varying sizes dispersed throughout the field of view. FIG. 15C illustrates the lyophilized agent loaded with a therapeutic agent, such as an immunoadjuvant, an anti-CD40 monoclonal antibody, or caflanone.

FIGS. 16A-16B illustrate transmission electron microscopy images of drug compounds, according to an embodiment. FIG. 16A illustrates a TEM image of the caflanone drug. The image displays a dispersed pattern of irregular, dark particles against a lighter background. FIG. 16B illustrates a TEM image of the anti-CD40 monoclonal antibody. In contrast to the caflanone image, this shows a single, well-defined spherical particle.

FIGS. 17A-17B illustrate imaging results of a biomaterial system over time, according to aspects of the present disclosure. FIG. 17A illustrates a series of CT images of a mouse at different time points are shown. The images display the full body of the mouse, with the skeletal structure clearly visible. FIG. 17B illustrates a series of MRI results of the same system are presented. The MRI images are divided into two rows: T1-Weighted and T1-Map.

FIG. 18 illustrates a series of CT images of a mouse skeleton over time, according to an embodiment.

FIGS. 19A-19C illustrate images of clonogenic assay dishes with varying concentrations of Caflanone from 1-10 μM Caflanone, additionally CLARITY biomaterial was tested. FIG. 19A illustrates graphical representation of the cell percent survival after treatment. Next clonogenic assays using KPC cell line were performed, images of clonogenic assay plates of KPC treated with radia-tion alone or radiation in combination with Caflanone. FIG. 19B illustrates the percent survival after treatment of radiation or adjuvanted with Caflanone, giving greater insight into the cell kill. Finally, we used human pan-creatic cancer cell line MIA PaCa2 and performed clonogenic assays, cells were treated with radiation alone in comparison to radiation and Caflanone immunoadjuvant. FIG. 19C illustrates respective percent cell survival after treatment comparing radiation alone with radiation and a Caflanone.

FIG. 20 illustrates transmission electron microscopy (right) and scanning electron microscopy (left) of CLARITY Biomaterial.

FIG. 21 illustrates T1 weighted images of No Treatment (top) Caflanone (middle) and CLARITY Biomaterial loaded with Caflanone (bottom).

FIGS. 22A-22F illustrate tumor growth and survival. FIG. 22A illustrates comparisons of the different treatment groups showing tumor growth in males. FIG. 22B illustrates comparisons of the different treatment groups showing survival in males. FIG. 22C illustrates comparisons of the different treatment groups showing tumor growth in females. FIG. 22D illustrates comparisons of the different treatment groups showing survival in females. Superior survival was shown when loading CLARITY Biomaterial with Caflanone as opposed to Caflanone alone in both male and female mice. Additionally, improved tumor control was shown in female mice when using the CLARITY Biomaterial and Caflanone. FIG. 22E illustrates flow cytometry data of CD4 and CD8 markers 23 days post treatment. FIG. 22F illustrates flow cytometry data of CD4 and CD8 markers 43 days post treatment. At 23 days post treatment greater lymphocyte statistics in the CLARITY Biomaterial and Caflanone than no treatment. These results give insight to improve the immune response in immunologically cold tumors.

### DETAILED DESCRIPTION OF THE INVENTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In the present disclosure an average of 53.9% reduction in cell proliferation was observed for KPC cells treated with either radiation alone or radiation and caflanone. Higher CD4 and CD8 T-cells infiltrations were observed for the mice tumors treated with CLARITY Biomaterial loaded with Caflanone compared to individual treatments or the control groups. Additionally improved tumor control was exhibited treating tumor bearing mice with CLARITY Biomaterial loaded with Caflanone.

Described herein is the use of flavonoid based drugs Caflanone-FBL-03G for imaging and treating pancreatic cancer. Caflanone is a non-cannabinoid metabolite of Cannabis Sativa which has demonstrated anti-inflammatory properties as well an anticancer drug because it has been shown to induce apoptosis and arrest part of the cancer cell cycle. Caflanone has previously been studied in pancreatic cancer models and has shown significant impact in being able to reduce tumor growth. However, little is known about the ability for Caflanone to invoke the immune response in vivo. As shown herein, caflanone was evaluated at different concentrations to test drug toxicity to fibroblast cells to establish a drug concentration of Caflanone that limits toxicity to normal cells. Then clonogenic assays were performed to test the efficacy of decreasing cell survival in pancreatic cancer cells, both murine and human, from caflanone. In conjunction with treating the cells with Caflanone alone cells were treated with radiation and the drugs to further understand the benefits of combining radiation and immunoadjuvants. Additionally, in vivo tumor control and overall survival while using the drugs alone as well as the CLARITY Biomaterial loaded with caflanone was evaluated. CLARITY biomaterial is a smart radiotherapy biomaterial which utilizes lyophilized magnesium oxide nanoparticles or gold nanoparticles, which are imageable on both T1-weighted and CT imaging and can be loaded with immunoadjuvants at the point of care. The significance of this work is testing the efficacy of flavonoid based drugs in-vitro and in vivo as well as investigating the further use of CLARITY biomaterial loaded with flavonoid based drugs.

The term “immunoadjuvant” refers to a substance that enhances the body's immune response to an antigen. Immunoadjuvant may stimulate stronger, longer-lasting immunity. Immunoadjuvants can work by various mechanisms, including but not limited to promoting antigen uptake by immune cells, activating innate immune pathways, or prolonging antigen presentation. Examples of immunoadjuvants include but are not limited to Aluminum salts (e.g., aluminum hydroxide, aluminum phosphate, potassium alum Calcium phosphate hydroxide), squalene, paraffin oil, adjuvant 65, propolis, killed bacteria (e.g., Bordetella pertussis, Mycobacterium bovis, toxoids, monophosphoryl lipid A (MPL), saponins, QS-21, interleukins (e.g., IL-1, IL-2, IL-12), CpG oligonucleotides (e.g., CpG 1018), Freund's complete adjuvant, Freund's incomplete adjuvant (e.g., oil-in-water emulsion), AS01 (MPL+QS-21), and matrix-M (QS-21+cholesterol+phospholipids).

The present disclosure provides a customizable lyophilized agent for radiotherapy imaging and therapy (CLARITY). This agent, which may be referred to as CLARITY Biomaterial, is designed to serve dual functions as a fiducial marker and a drug delivery system. The CLARITY Biomaterial comprises a biocompatible lyophilized matrix that can encapsulate a range of therapeutic agents to enhance therapy. The lyophilized matrix can be reconstituted with a biocompatible solvent to form a colloid, enabling easy administration. The CLARITY Biomaterial also incorporates nanoparticles that provide imaging contrast for at least one imaging modality, such as computed tomography (CT) or magnetic resonance imaging (MRI), thereby facilitating image-guided radiotherapy.

In some embodiments are directed to a customizable lyophilized agent, including: a biocompatible lyophilized matrix; and nanoparticles incorporated within the lyophilized matrix, wherein the lyophilized matrix is reconstitutable with a biocompatible solvent to form a colloid, and wherein the nanoparticles provide imaging contrast for at least one imaging modality.

In some embodiments are directed to a method of imaging a subject using a customizable lyophilized agent, including: reconstituting a lyophilized agent with a biocompatible solvent to form a colloid, wherein the lyophilized agent comprises a biocompatible lyophilized matrix and nanoparticles incorporated within the lyophilized matrix; administering the colloid to the subject; and imaging the subject using at least one imaging modality, wherein the nanoparticles provide imaging contrast for the at least one imaging modality.

In some embodiments are directed to a method of treatment using image-guided radiation therapy (IGRT) including administering to the subject the customizable lyophilized agent as disclosed herein.

The term “subject” as used herein refers to any individual or patient to which the subject methods are performed. Generally, the subject is human, although as will be appreciated by those in the art, the subject may be a non-human animal. Thus other animals, including vertebrate such as rodents (including mice, rats, hamsters and guinea pigs), cats, dogs, rabbits, farm animals including cows, horses, goats, sheep, pigs, chickens, and non-human primates (including monkeys, chimpanzees, orangutans and gorillas) are included within the definition of subject.

The term “treatment” is used interchangeably herein with the term “therapeutic method” or “therapy” and refers to 1) therapeutic treatments or measures that cure, slow down, lessen symptoms of, and/or halt progression of a diagnosed pathologic conditions or disorder, and/or 2) prophylactic/preventative measures. Those in need of treatment may include individuals already having a particular medical disorder as well as those who may ultimately acquire the disorder (i.e., those needing preventive measures).

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration.

Exemplary routes of administration include but are not limited to inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracoronal, intracoronary, intracorpous cavernaosum, intradermal, intradiscal, intraductal, intraduodenal, intradural, intraepidermal, intraesophageal, intragastric, intragingival, intrahippocampal, intraileal, intralesional, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic administration, or any combination thereof.

In some embodiments are directed to a method of preparing a customizable lyophilized agent, including: mixing a biocompatible hydrogel with nanoparticles to form a mixture; freezing the mixture; and lyophilizing the frozen mixture to produce a lyophilized agent, wherein the lyophilized agent is reconstitutable with a biocompatible solvent to form a colloid, and wherein the nanoparticles provide imaging contrast for at least one imaging modality.

The term “lyophilized agent” refers to a pharmaceutical or biologically active compound that has undergone lyophilization. Lyophilization can be done by any method known in the art including but not limited to freeze-drying (e.g., freezing the compound and subsequently removing water or solvent by sublimation under reduced pressure), resulting in a dry, stable product. The lyophilized agent may include active ingredients, excipients, stabilizers, or bulking agents, and is formulated to retain biological activity and structural integrity upon reconstitution as described herein. Methods of lyophilization include but are not limited to manifold drying, batch drying, and bulk drying.

In some aspects, the CLARITY Biomaterial may be designed to be affordable and easy to use, making it suitable for various clinical settings, including low-resource environments. The lyophilized form of the CLARITY Biomaterial may offer advantages such as increased shelf-life and stability, ease of handling and implantation, reduced risk of marker migration, and facilitated transport and storage. Furthermore, the CLARITY Biomaterial may be customizable to contain one or more therapeutic drugs, allowing it to serve a dual purpose as both a fiducial marker and a drug delivery system. This dual functionality may enhance therapy and reduce toxicity by delivering drugs directly to the treatment site.

In some embodiments, the lyophilized agent may also be loaded with drugs in situ at the tumor site. The drug delivery capability of the lyophilized agent may enhance therapy outcomes by delivering therapeutic agents directly to the treatment site, thereby reducing systemic side effects.

The nanoparticles in the lyophilized agent provide imaging contrast for the at least one imaging modality, facilitating accurate and precise targeting of the tumor site during radiotherapy.

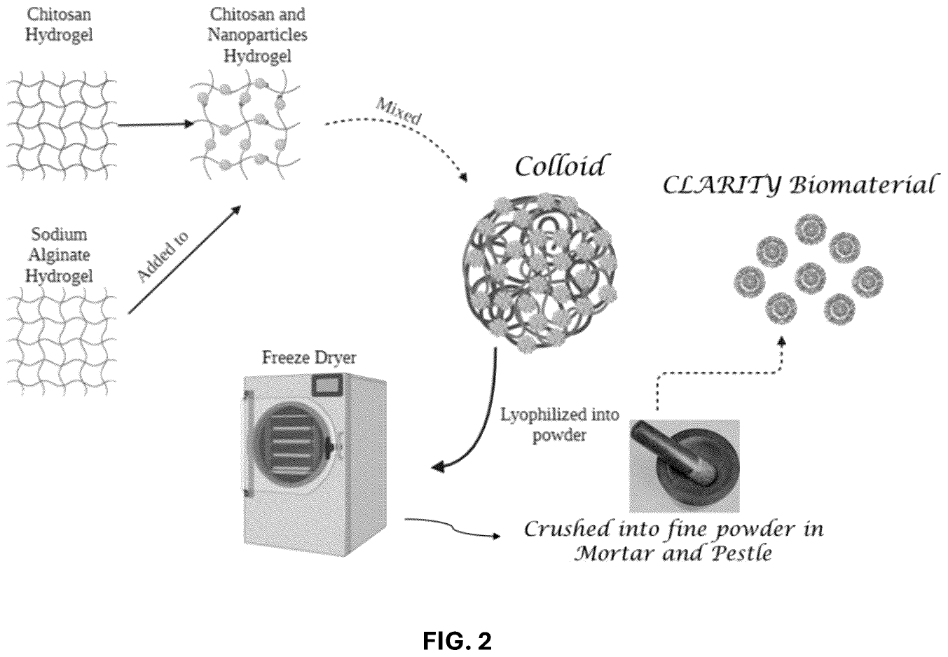

Referring to FIG. 2, the process of producing the customizable lyophilized agent is further illustrated.

Referring to FIG. 2, the process of reconstituting the lyophilized agent at the point of care is further illustrated.

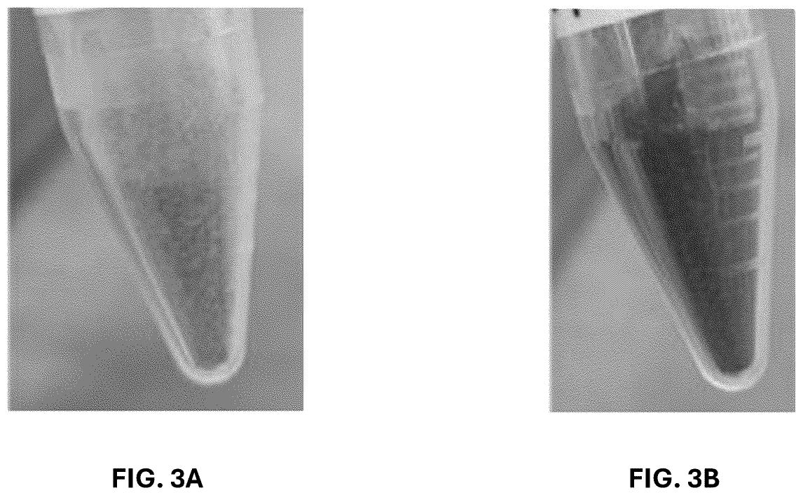

Referring to FIG. 3A and FIG. 3B, the customizable lyophilized agent is shown in its natural form. For instance, FIG. 3A illustrates a lyophilized agent formulated with gold nanoparticles (GNP), while FIG. 3B depicts a lyophilized agent formulated with manganese oxide nanoparticles (Mn₂O₃).

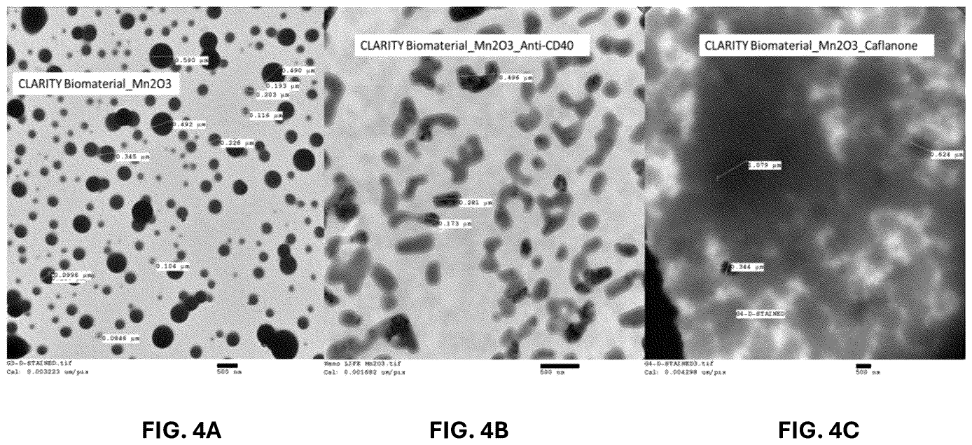

Referring to FIG. 4A-4C, transmission electron microscopy (TEM) images of the customizable lyophilized agent in different formulations are illustrated.

FIG. 4A depicts a TEM image of the lyophilized agent formulated with manganese oxide (Mn₂O₃) nanoparticles. The image reveals a distribution of spherical nanoparticles with varying sizes, dispersed throughout a less dense background matrix. The nanoparticles appear as darker spots against the lighter background, indicating their solid nature in the lyophilized matrix.

FIG. 4B shows a TEM image of the lyophilized agent (Mn₂O₃) loaded with caflanone. This image reveals a denser distribution of nanoparticles compared to FIG. 4A. The nanoparticles appear to be more closely packed, with some areas showing aggregation or clustering of particles. This suggests that the addition of the caflanone drug to the lyophilized agent may result in changes in the distribution and aggregation of the nanoparticles within the matrix.

FIG. 4C presents a TEM image of the lyophilized agent (Mn₂O₃) loaded with Anti-CD40. This image shows a distribution of nanoparticles similar to FIG. 4B, but with some distinct differences in particle arrangement and density. There appears to be more variation in particle size and some areas of the image show larger, potentially aggregated structures. This indicates that the incorporation of the Anti-CD40 monoclonal antibody into the lyophilized agent may result in different morphological changes compared to the caflanone-loaded agent.

These TEM images provide visual evidence of the nanostructure of the lyophilized agent and how it changes when loaded with different therapeutic agents. The images reveal details about the morphology and size distribution of the nanoparticles within the lyophilized matrix, providing insights into the structural characteristics of the agent in different states. This information may be useful for understanding the behavior of the lyophilized agent in various applications, such as image-guided radiotherapy and drug delivery.

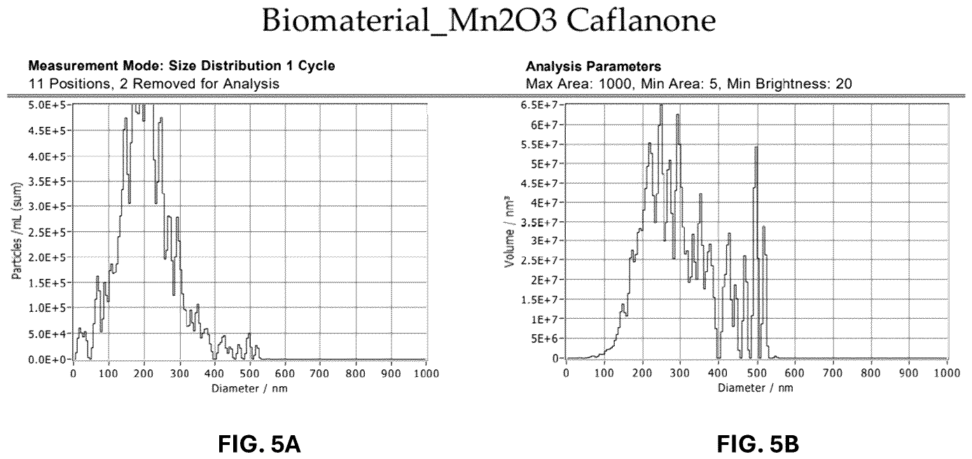

Referring to FIG. 5A and FIG. 5B, the size distribution of the customizable lyophilized agent formulated with manganese oxide nanoparticles and loaded with caflanone is illustrated. In some aspects, the size distribution of the lyophilized agent may be analyzed using nanoparticle tracking analysis (NTA), a technique that allows for the measurement of the size and concentration of nanoparticles in a suspension. The NTA may provide information about the size distribution of the nanoparticles in the lyophilized agent, which may be useful for understanding the behavior of the agent in various applications, such as image-guided radiotherapy and drug delivery.

FIG. 5A depicts a particle concentration distribution graph for the lyophilized agent. The graph displays particle concentration in particles/mL on the y-axis and particle diameter in nanometers on the x-axis. The graph shows a multimodal distribution with peaks around 100-300 nm, indicating the presence of nanoparticles of varying sizes in the lyophilized agent. This size distribution may be a result of the formulation process of the lyophilized agent, which may involve the incorporation of nanoparticles of different sizes into the biocompatible lyophilized matrix.

FIG. 5B presents a volume distribution graph for the same lyophilized agent. The graph displays volume in nm³ on the y-axis and particle diameter in nanometers on the x-axis. The graph also shows a multimodal distribution with prominent peaks in the 200-400 nm range. This volume distribution may reflect the varying sizes of the nanoparticles in the lyophilized agent, as well as the potential aggregation or clustering of nanoparticles within the matrix.

The size and volume distribution of the nanoparticles in the lyophilized agent may affect the imaging contrast provided by the agent, as well as the release and delivery of the therapeutic agent loaded into the matrix. For instance, larger nanoparticles may provide stronger imaging contrast, while smaller nanoparticles may allow for a more controlled and sustained release of the therapeutic agent. Therefore, the size and volume distribution of the nanoparticles in the lyophilized agent may be tailored to achieve desired imaging and therapeutic outcomes.

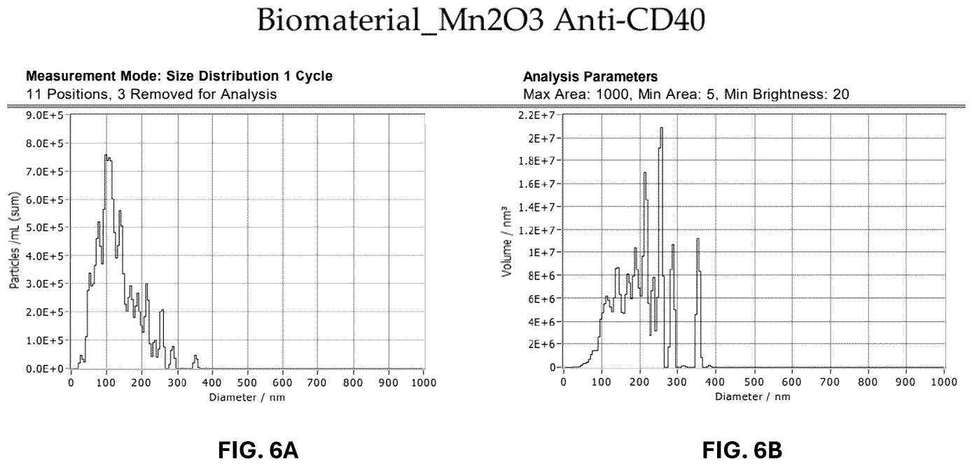

Referring to FIG. 6A and FIG. 6B, the size distribution analysis of the customizable lyophilized agent formulated with manganese oxide nanoparticles and loaded with anti-CD40 antibody is illustrated. In some aspects, the size distribution of the lyophilized agent may be analyzed using nanoparticle tracking analysis (NTA), a technique that allows for the measurement of the size and concentration of nanoparticles in a suspension. The NTA may provide information about the size distribution of the nanoparticles in the lyophilized agent, which may be useful for understanding the behavior of the agent in various applications, such as image-guided radiotherapy and drug delivery.

FIG. 6A depicts a particle concentration distribution graph for the lyophilized agent. The graph displays particle concentration in particles/mL on the y-axis and particle diameter in nanometers on the x-axis. The graph shows a multimodal distribution with peaks around 100-300 nm, indicating the presence of nanoparticles of varying sizes in the lyophilized agent. This size distribution may be a result of the formulation process of the lyophilized agent, which may involve the incorporation of nanoparticles of different sizes into the biocompatible lyophilized matrix.

FIG. 6B presents a volume distribution graph for the same lyophilized agent. The graph displays volume in nm³ on the y-axis and particle diameter in nanometers on the x-axis. The graph also shows a multimodal distribution with prominent peaks in the 200-400 nm range. This volume distribution may reflect the varying sizes of the nanoparticles in the lyophilized agent, as well as the potential aggregation or clustering of nanoparticles within the matrix.

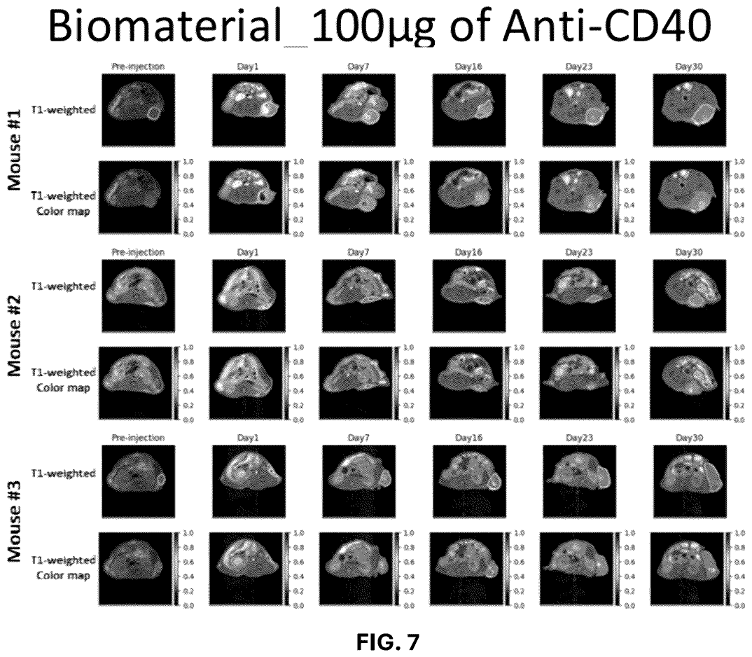

Referring to FIG. 7, the MRI imaging results of mice injected with the customizable lyophilized agent containing anti-CD40 antibody over a 30-day period are illustrated. In some aspects, the lyophilized agent may be formulated with manganese oxide nanoparticles and loaded with anti-CD40 antibody. The agent may be administered to a subject, such as a mouse, and the subject may be imaged using at least one imaging modality, such as MRI, over a period of time. The nanoparticles in the lyophilized agent provide imaging contrast for the imaging modality, facilitating accurate and precise targeting of the tumor site during radiotherapy.

The imaging results may show the visibility and persistence of the lyophilized agent as a fiducial marker over the 30-day period. The imaging contrast provided by the nanoparticles may be visible from the first day post-injection and may persist for up to 30 days post-injection. This long-term visibility of the lyophilized agent may facilitate its use as a fiducial marker in image-guided radiotherapy, allowing for accurate and precise targeting of the tumor site throughout the course of treatment.

The imaging results may also show changes in the signal intensity or contrast of the lyophilized agent over time. These changes may be due to the release and distribution of the therapeutic agent loaded into the lyophilized agent, as well as the degradation or clearance of the agent from the body. The changes in signal intensity or contrast over time may provide additional information about the behavior of the lyophilized agent in vivo, such as its drug release kinetics, biodistribution, and clearance rate.

The imaging results may be used to monitor the treatment response and assess the efficacy of the lyophilized agent in enhancing therapy outcomes. For instance, changes in the size or signal intensity of the tumor site may indicate the therapeutic effect of the therapeutic agent delivered by the lyophilized agent. The imaging results may also be used to adjust the treatment plan or dosing regimen, if necessary, to optimize the therapeutic efficacy and minimize potential side effects.

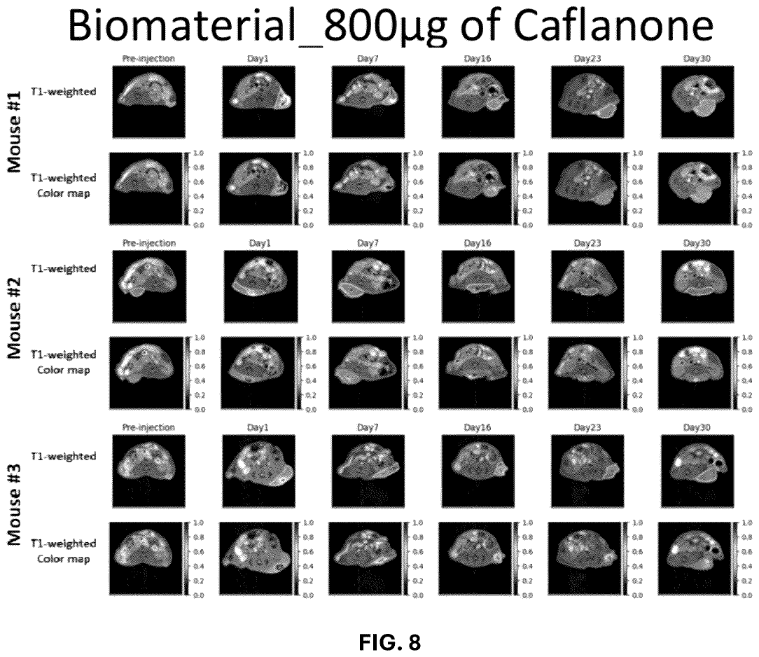

Referring to FIG. 8, the MRI imaging results of mice injected with the customizable lyophilized agent containing caflanone over a 30-day period are illustrated. In some aspects, the lyophilized agent may be formulated with manganese oxide nanoparticles and loaded with caflanone. The agent may be administered to a subject, such as a mouse, and the subject may be imaged using at least one imaging modality, such as MRI, over a period of time. The nanoparticles in the lyophilized agent provide imaging contrast for the imaging modality, facilitating accurate and precise targeting of the tumor site during radiotherapy.

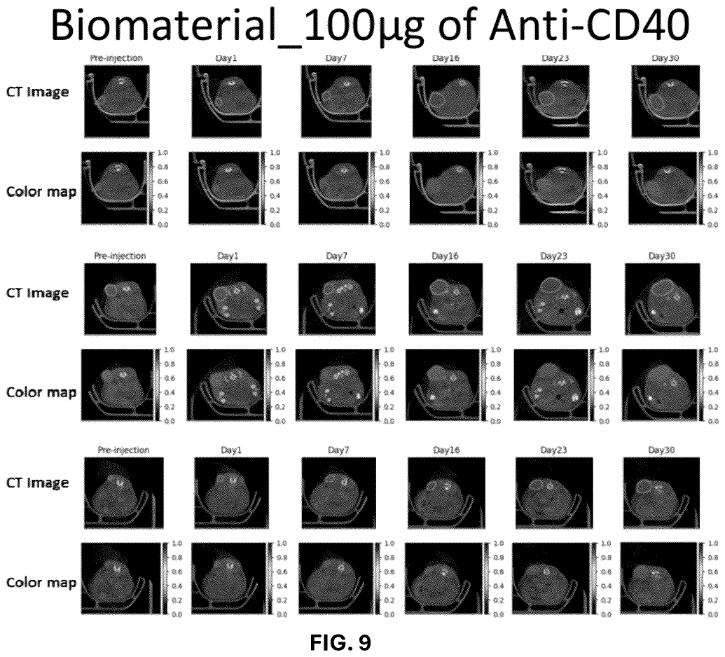

Referring to FIG. 9, the CT imaging results of mice injected with the customizable lyophilized agent containing anti-CD40 antibody over a 30-day period are illustrated. In some aspects, the lyophilized agent may be formulated with manganese oxide nanoparticles and loaded with anti-CD40 antibody. The agent may be administered to a subject, such as a mouse, and the subject may be imaged using at least one imaging modality, such as CT, over a period of time. The nanoparticles in the lyophilized agent provide imaging contrast for the imaging modality, facilitating accurate and precise targeting of the tumor site during radiotherapy.

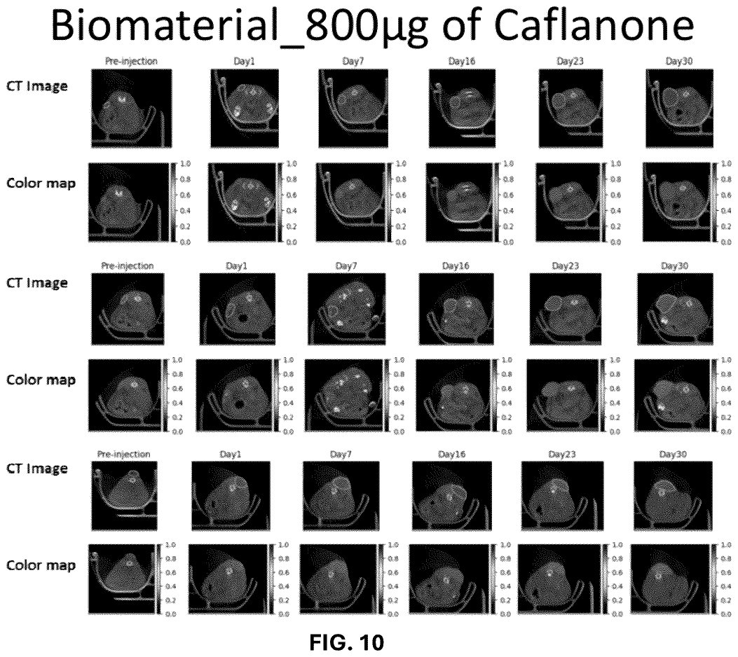

Referring to FIG. 10, the CT imaging results of mice injected with the customizable lyophilized agent containing caflanone over a 30-day period are illustrated. In some aspects, the lyophilized agent may be formulated with manganese oxide nanoparticles and loaded with caflanone. The agent may be administered to a subject, such as a mouse, and the subject may be imaged using at least one imaging modality, such as CT, over a period of time. The nanoparticles in the lyophilized agent provide imaging contrast for the imaging modality, facilitating accurate and precise targeting of the tumor site during radiotherapy.

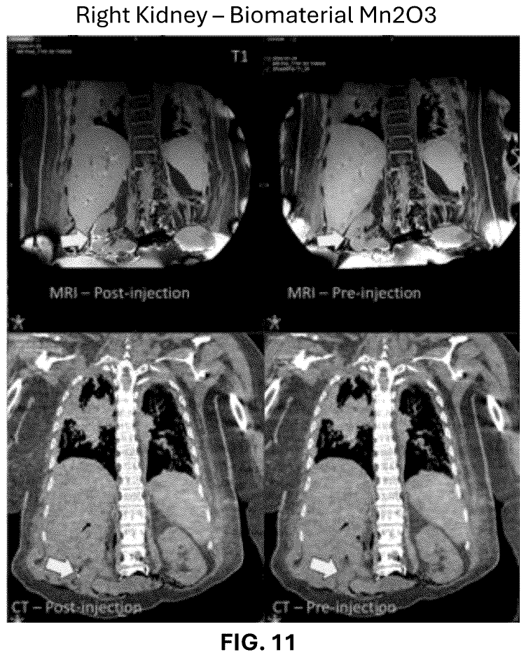

Referring to FIG. 11, the MRI and CT imaging results of a human cadaver's right kidney before and after injection of the customizable lyophilized agent are illustrated. In some aspects, the lyophilized agent may be formulated with manganese oxide nanoparticles and loaded with a therapeutic agent. The agent may be administered to a subject, such as a human cadaver, and the subject may be imaged using at least one imaging modality, such as MRI or CT, over a period of time. The nanoparticles in the lyophilized agent provide imaging contrast for the imaging modality, facilitating accurate and precise targeting of the treatment site during radiotherapy.

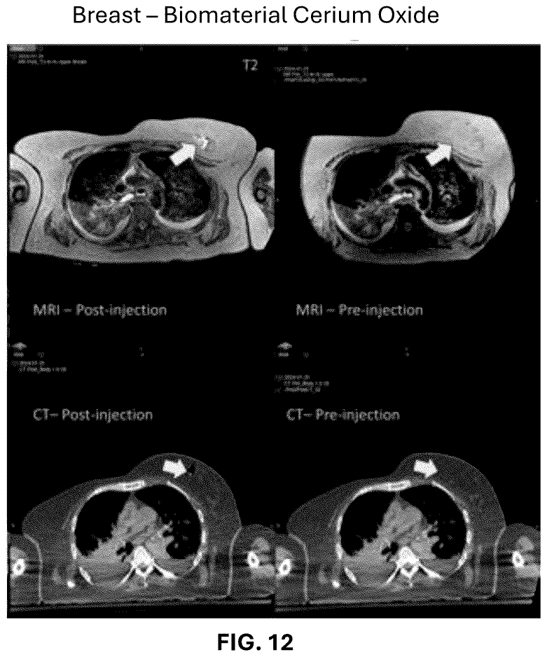

Referring to FIG. 12, the MRI and CT imaging results of a human cadaver's breast tissue before and after injection of the customizable lyophilized agent containing cerium oxide nanoparticles are illustrated. In some aspects, the lyophilized agent may be formulated with cerium oxide nanoparticles and loaded with a therapeutic agent. The agent may be administered to a subject, such as a human cadaver, and the subject may be imaged using at least one imaging modality, such as MRI or CT, over a period of time. The nanoparticles in the lyophilized agent provide imaging contrast for the imaging modality, facilitating accurate and precise targeting of the treatment site during radiotherapy.

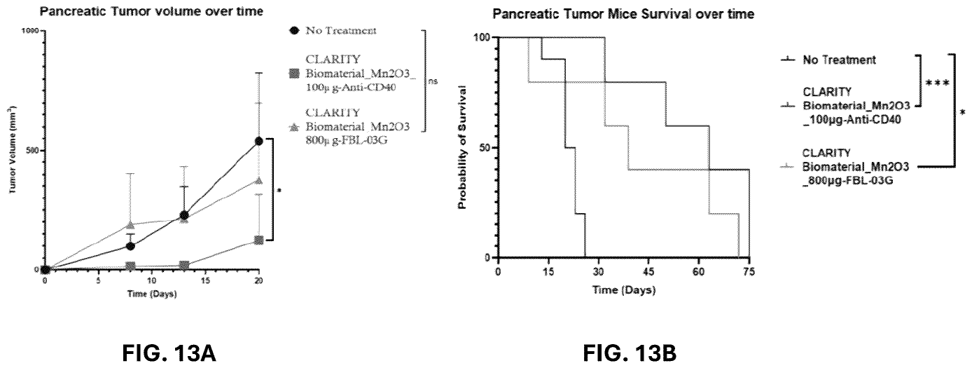

Referring to FIG. 13A and FIG. 13B, the efficacy results of the customizable lyophilized agent in pancreatic cancer treatment are illustrated. In some aspects, the lyophilized agent may be formulated with manganese oxide nanoparticles and loaded with either anti-CD40 antibody or caflanone. The agent may be administered to a subject, such as a mouse, and the therapeutic response may be monitored over a period of time.

FIG. 13A depicts a graph showing the changes in pancreatic tumor volume over time for different treatment groups. The graph displays tumor volume in mm³ on the y-axis and time in days on the x-axis. Three treatment conditions are compared: no treatment, CLARITY Biomaterial Mn₂O₃ with 100 μg Anti-CD40, and CLARITY Biomaterial Mn₂O₃ with 800 μg FBL-03G. In some cases, the graph may show a significant delay in tumor growth for the group treated with the lyophilized agent loaded with anti-CD40 compared to the no treatment group. This suggests that the lyophilized agent may enhance therapy outcomes by delivering the anti-CD40 antibody directly to the tumor site.

FIG. 13B illustrates a survival curve for the same treatment groups over the same period. The graph shows the probability of survival on the y-axis and time in days on the x-axis. In some aspects, the survival curve may show a significant increase in survival for the group treated with the lyophilized agent loaded with anti-CD40 or caflanone compared to the no treatment group. This suggests that the lyophilized agent may not only enhance therapy outcomes but also improve survival rates in pancreatic cancer treatment.

These efficacy results provide evidence of the therapeutic potential of the customizable lyophilized agent in enhancing therapy outcomes and improving survival rates in pancreatic cancer treatment. The results also demonstrate the versatility of the lyophilized agent in being able to be loaded with different therapeutic agents, such as anti-CD40 antibody or caflanone, to achieve desired therapeutic effects.

The term “flavonoid” refers to a diverse group of naturally occurring plant compounds known for their antioxidant, anti-inflammatory, and potential disease-preventing properties. Examples of flavonoid include but are not limited to flavonols (e.g., quercetin, myricetin, fisetin), flavones (e.g., apigenin, luteolin), caflanone, flavanones (e.g., hesperidin, naringenin), flavan-3-ols (catechins) (e.g., epicatechin, epigallocatechin gallate (EGCG), anthocyanins (e.g., cyanidin, delphinidin, malvidin), isoflavones (e.g., genistein, daidzein), chalcones.

The term “caflanone” or “FBL-03G” refers to an experimental small-molecule drug. It is derived from a rare Cannabis strain called Black Swan and belongs to a class of compounds known as flavonoids, specifically prenylated phenolic flavones.

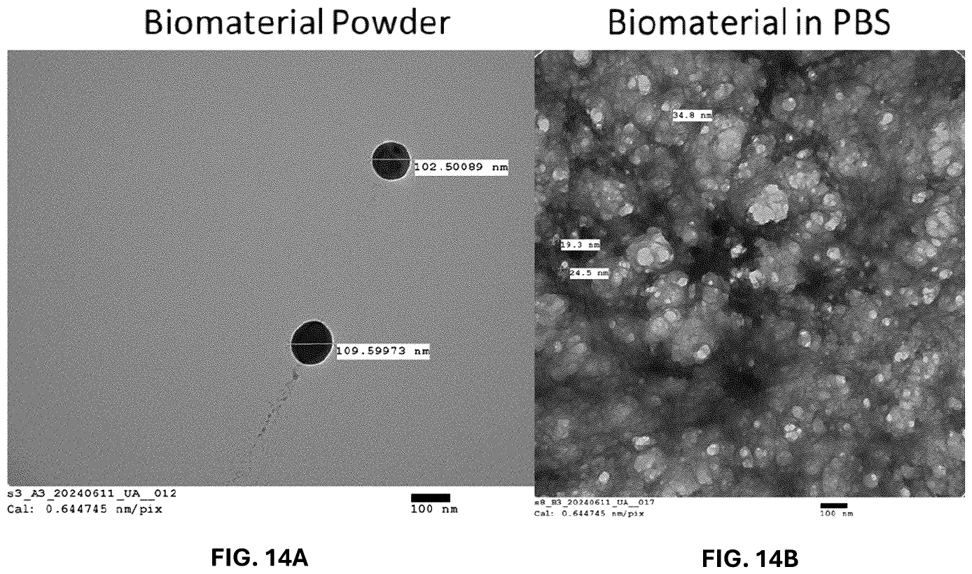

Referring to FIG. 14A and FIG. 14B, transmission electron microscopy (TEM) images of the customizable lyophilized agent in different states are illustrated. In some aspects, the lyophilized agent may be in a powder form, as shown in FIG. 14A, or it may be suspended in a biocompatible solvent, such as phosphate-buffered saline (PBS), as shown in FIG. 14B. The TEM images provide visual information on the morphology and size distribution of the lyophilized agent in these different states.

In FIG. 14A, the TEM image of the lyophilized agent in powder form reveals a distribution of spherical nanoparticles with varying sizes, dispersed throughout a less dense background matrix. The nanoparticles appear as darker spots against the lighter background, indicating their solid nature in the powder form. The size and morphology of the nanoparticles in the powder form may be influenced by the lyophilization process, which involves the removal of water from the frozen mixture by sublimation and desorption.

In FIG. 14B, the TEM image of the lyophilized agent suspended in PBS shows a more complex structure, with numerous particles of varying sizes dispersed throughout the field of view. The suspended lyophilized agent forms a network of interconnected particles, creating a heterogeneous appearance. The size and morphology of the particles in the suspension may be influenced by the reconstitution process, which involves the addition of the biocompatible solvent to the lyophilized agent, followed by mixing or agitation to form a homogeneous colloid.

The comparison between the TEM images of the lyophilized agent in powder form and in suspension provides insights into the structural changes that occur when the lyophilized agent transitions from a dry powder to a suspended state. These structural changes may be related to the lyophilized agent's intended functionality in different environments. For instance, in its powder form, the lyophilized agent may offer advantages such as increased shelf-life and stability, ease of handling and implantation, and facilitated transport and storage. On the other hand, in its suspended state, the lyophilized agent may provide a homogeneous colloid that is easy to administer and capable of delivering therapeutic agents directly to the treatment site.

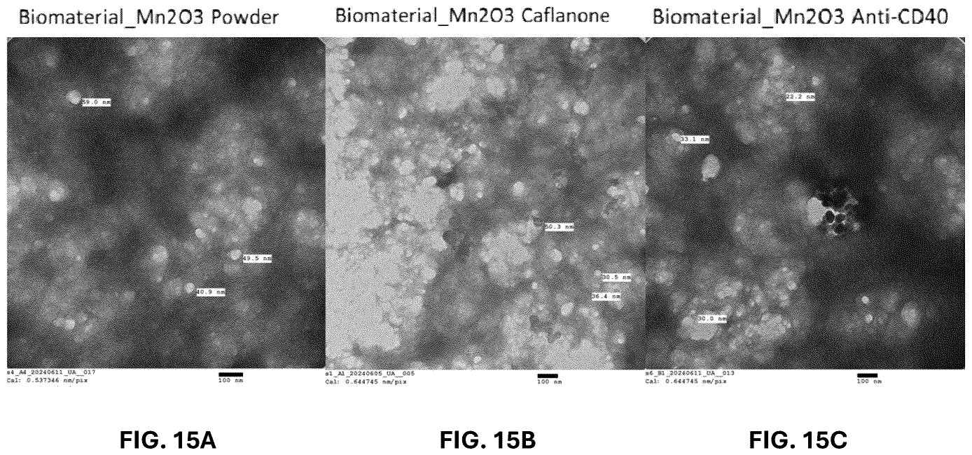

Referring to FIG. 15A, FIG. 15B, and FIG. 15C, transmission electron microscopy (TEM) images of the customizable lyophilized agent formulated with manganese oxide nanoparticles in different states are illustrated. In some aspects, the lyophilized agent may be in a powder form, as shown in FIG. 15A, or it may be suspended in a biocompatible solvent, such as phosphate-buffered saline (PBS), as shown in FIG. 15B. The TEM images provide visual information on the morphology and size distribution of the lyophilized agent in these different states.

In FIG. 15A, the TEM image of the lyophilized agent in powder form reveals a distribution of spherical nanoparticles with varying sizes, dispersed throughout a less dense background matrix. The nanoparticles appear as darker spots against the lighter background, indicating their solid nature in the powder form. The size and morphology of the nanoparticles in the powder form may be influenced by the lyophilization process, which involves the removal of water from the frozen mixture by sublimation and desorption.

In FIG. 15B, the TEM image of the lyophilized agent suspended in PBS shows a more complex structure, with numerous particles of varying sizes dispersed throughout the field of view. The suspended lyophilized agent forms a network of interconnected particles, creating a heterogeneous appearance. The size and morphology of the particles in the suspension may be influenced by the reconstitution process, which involves the addition of the biocompatible solvent to the lyophilized agent, followed by mixing or agitation to form a homogeneous colloid.

In some embodiments, the lyophilized agent may be loaded with a therapeutic agent, such as an immunoadjuvant, an anti-CD40 monoclonal antibody, or caflanone, as shown in FIG. 15C. The therapeutic agent may be incorporated within the lyophilized matrix during the reconstitution process, allowing the lyophilized agent to serve a dual purpose as both a fiducial marker and a drug delivery system. The TEM image of the lyophilized agent loaded with the therapeutic agent reveals a distribution of nanoparticles similar to that observed in FIG. 15B, but with some distinct differences in particle arrangement and density. There appears to be more variation in particle size and some areas of the image show larger, potentially aggregated structures. This indicates that the incorporation of the therapeutic agent into the lyophilized agent may result in different morphological changes compared to the agent in its powder form or suspended state.

These TEM images provide visual evidence of the nanostructure of the lyophilized agent and how it changes when loaded with different therapeutic agents. The images reveal details about the morphology and size distribution of the nanoparticles within the lyophilized matrix, providing insights into the structural characteristics of the agent in different states. This information may be useful for understanding the behavior of the lyophilized agent in various applications, such as image-guided radiotherapy and drug delivery.

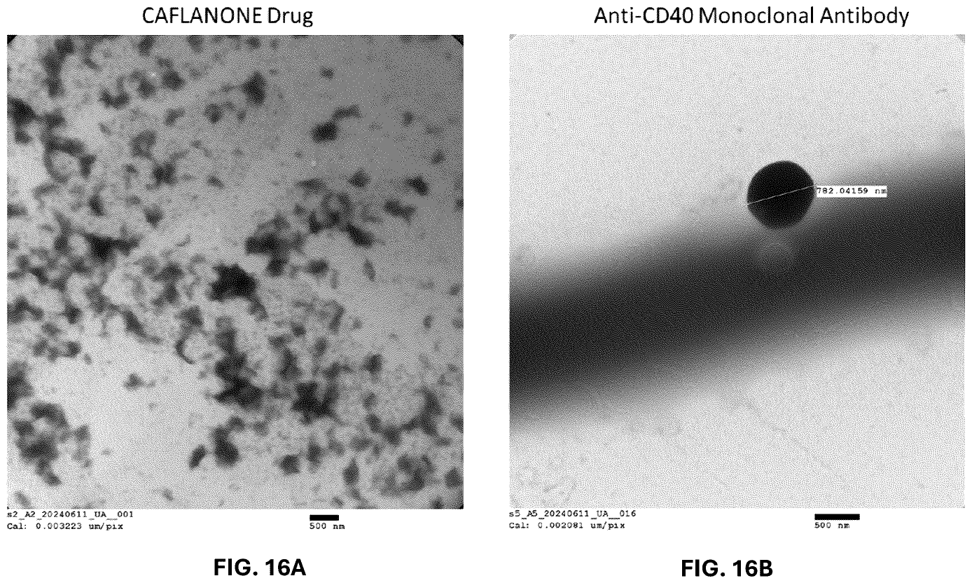

Referring to FIG. 16A and FIG. 16B, transmission electron microscopy (TEM) images of two different therapeutic agents, caflanone and anti-CD40 monoclonal antibody, are illustrated. These therapeutic agents may be incorporated within the lyophilized matrix of the customizable lyophilized agent during the reconstitution process, allowing the lyophilized agent to serve a dual purpose as both a fiducial marker and a drug delivery system. The therapeutic agent may comprise one or more drugs, such as an immunoadjuvant, an anti-CD40 monoclonal antibody, or caflanone, which may be used to enhance therapy outcomes.

FIG. 16A depicts a TEM image of the caflanone drug. The image displays a dispersed pattern of irregular, dark particles against a lighter background. The particles vary in size and shape, with some appearing as small, discrete dots while others form larger, more amorphous clusters. This heterogeneous distribution suggests the caflanone drug forms aggregates or crystals of varying sizes when in solution or dried state.

FIG. 16B presents a TEM image of the anti-CD40 monoclonal antibody. In contrast to the caflanone image, this shows a single, well-defined spherical particle. The particle appears as a dark, circular object against a lighter background. A measurement line across the particle indicates its diameter is approximately 782.06159 nm. The uniform shape and size of this particle is consistent with the typical structure of monoclonal antibodies, which tend to form more homogeneous solutions.

These TEM images provide visual evidence of the nanostructure of the therapeutic agents and how they may be incorporated within the lyophilized matrix of the customizable lyophilized agent. The images reveal details about the morphology and size distribution of the therapeutic agents, providing insights into their structural characteristics and potential behavior when loaded into the lyophilized agent. This information may be useful for understanding the behavior of the lyophilized agent in various applications, such as image-guided radiotherapy and drug delivery.

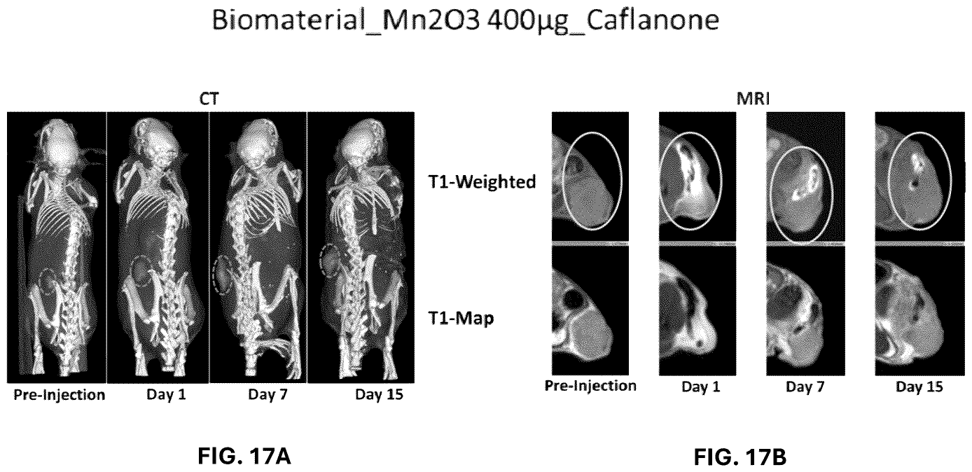

Referring to FIG. 17A and FIG. 17B, the imaging results of the customizable lyophilized agent containing manganese oxide nanoparticles and caflanone over a 15-day period are illustrated. In some aspects, the lyophilized agent may be formulated with manganese oxide nanoparticles and loaded with caflanone. The agent may be administered to a subject, such as a mouse, and the subject may be imaged using at least one imaging modality, such as computed tomography (CT) or magnetic resonance imaging (MRI), over a period of time. The nanoparticles in the lyophilized agent provide imaging contrast for the imaging modality, facilitating accurate and precise targeting of the tumor site during radiotherapy.

In FIG. 17A, a series of CT images of a mouse at different time points are shown. The images display the full body of the mouse, with the skeletal structure clearly visible. A blue dotted circle in the abdominal region indicates the location of interest where the biomaterial was injected. The imaging contrast provided by the nanoparticles may be visible from the first day post-injection and may persist for up to 15 days post-injection. This long-term visibility of the lyophilized agent may facilitate its use as a fiducial marker in image-guided radiotherapy, allowing for accurate and precise targeting of the tumor site throughout the course of treatment.

In FIG. 17B, a series of MRI results of the same system are presented. The MRI images are divided into two rows: T1-Weighted and T1-Map. The T1-Weighted images show a yellow circle highlighting the region of interest where the biomaterial was injected. The T1-Map images provide additional contrast information of the same area. Both sets of MRI images are shown at four time points: pre-injection, day 1, day 7, and day 15.

The imaging results demonstrate the long-term visibility of the biomaterial in MRI scans and its changing signal intensity over the 15-day period. This imaging system allows for tracking of the biomaterial's location and distribution over time, which can be useful for various biomedical applications such as drug delivery or tissue engineering. The dual-modality imaging capability of the system provides complementary information, enhancing its potential utility in research and clinical settings.

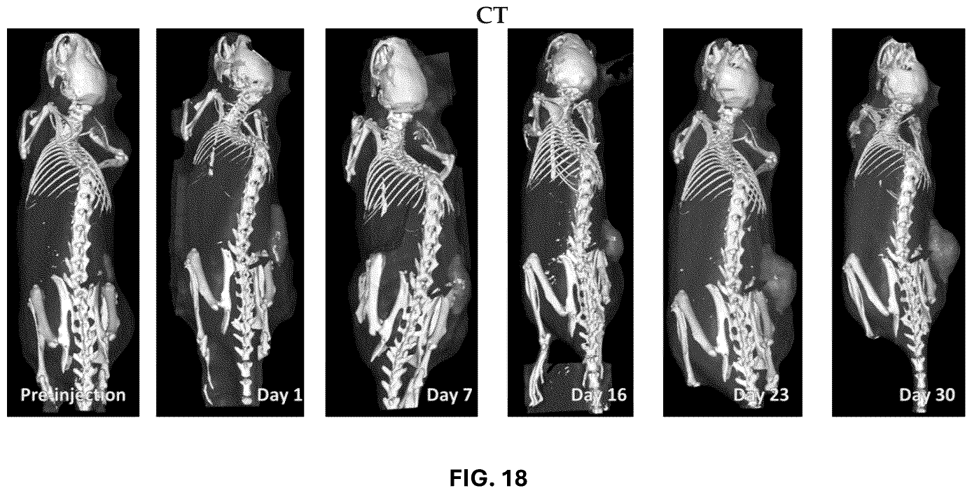

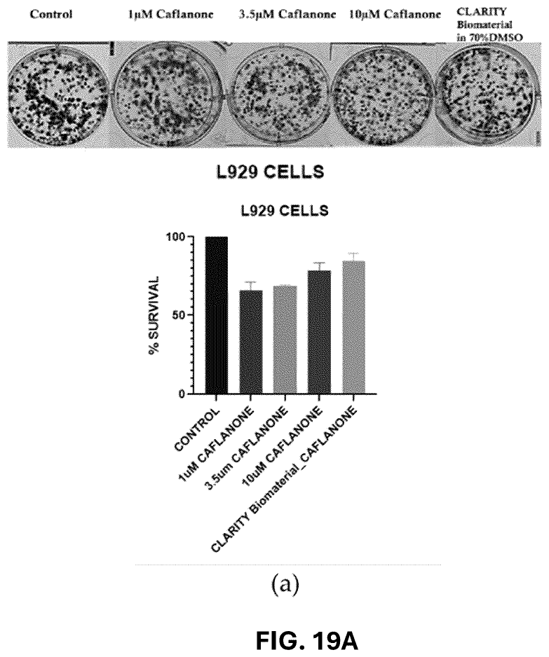

Referring to FIG. 18, the CT imaging results of a mouse injected with the customizable lyophilized agent over a 30-day period are illustrated. In some aspects, the lyophilized agent may be formulated with manganese oxide nanoparticles and loaded with a therapeutic agent. The agent may be administered to a subject, such as a mouse, and the subject may be imaged using at least one imaging modality, such as CT, over a period of time. The nanoparticles in the lyophilized agent provide imaging contrast for the imaging modality, facilitating accurate and precise targeting of the tumor site during radiotherapy.

Some embodiments are directed to a customizable lyophilized agent, comprising: a biocompatible lyophilized matrix; and nanoparticles incorporated within the lyophilized matrix, wherein the lyophilized matrix is reconstitutable with a biocompatible solvent to form a colloid, and wherein the nanoparticles provide imaging contrast for at least one imaging modality.

In some embodiments, the nanoparticles comprise at least one of manganese oxide nanoparticles, cerium oxide nanoparticles, gold nanoparticles, titanium dioxide nanoparticles, or gadodiamide nanoparticles.

In some embodiments, the nanoparticles provide imaging contrast for computed tomography (CT), magnetic resonance imaging (MRI), or a combination thereof.

In some embodiments, the biocompatible lyophilized matrix comprises chitosan and sodium alginate.

In some embodiments, the customizable lyophilized agent further comprises a therapeutic agent incorporated within the lyophilized matrix.

In some embodiments, the therapeutic agent is an antibody, an immunoadjuvant, a small molecule drug, a nucleic acid, a peptide, or a combination thereof.

In some embodiments, the therapeutic agent is an immunoadjuvant.

In some embodiments, the antibody is an anti-CD40 monoclonal antibody.

In some embodiments, the small molecule drug is caflanone.

In some embodiments, the lyophilized matrix is reconstitutable with an aqueous solution of the therapeutic agent.

Some embodiments are directed to a method of imaging a subject using a customizable lyophilized agent, comprising: reconstituting a lyophilized agent with a biocompatible solvent to form a colloid, wherein the lyophilized agent comprises a biocompatible lyophilized matrix and nanoparticles incorporated within the lyophilized matrix; administering the colloid to the subject; and imaging the subject using at least one imaging modality, wherein the nanoparticles provide imaging contrast for the at least one imaging modality.

In some embodiments, the at least one imaging modality comprises computed tomography (CT), magnetic resonance imaging (MRI), or a combination thereof.

In some embodiments, the nanoparticles comprise at least one of manganese oxide nanoparticles, cerium oxide nanoparticles, gold nanoparticles, titanium dioxide nanoparticles, or gadodiamide nanoparticles.

In some embodiments, the method further comprises administering radiotherapy to the subject based on imaging data obtained from the at least one imaging modality.

In some embodiments, the biocompatible solvent comprises an aqueous solution of a therapeutic agent.

In some embodiments, the therapeutic agent is an antibody, an immunoadjuvant, a small molecule drug, a nucleic acid, a peptide, or a combination thereof.

In some embodiments, the therapeutic agent is an immunoadjuvant.

In some embodiments, the antibody is an anti-CD40 monoclonal antibody.

In some embodiments, the small molecule drug is caflanone.

Some embodiments are directed to a method of treatment using image-guided radiation therapy (IGRT) comprising administering to the subject the customizable lyophilized agent of claim 1.

In some embodiments, the treatment is for cancer.

In some embodiments, the cancer is arteriovenous malformations, bone cancer, brain cancer, breast cancer, chondrosarcoma, chordoma, colon cancer, esophageal cancer, Ewing's sarcoma, head and neck cancers, leukemia, liver cancer, lung cancer, lymphoma, metastatic brain cancer, meningioma, neuroma, non-Hodgkin's lymphoma, osteosarcoma, pancreatic cancer, paranasal sinus cancer, prostate cancer, soft tissue sarcomas, spinal cord tumors, trigeminal neuralgia, or a combination thereof.

In some embodiments, the cancer is pancreatic cancer.

In some embodiments, the method further comprises administering a therapeutic agent.

In some embodiments, the therapeutic agent is an antibody, an immunoadjuvant, a small molecule drug, a nucleic acid, a peptide, or a combination thereof.

In some embodiments, the therapeutic agent is an immunoadjuvant.

In some embodiments, the antibody is an anti-CD40 monoclonal antibody. In some embodiments, the small molecule drug is caflanone.

In some embodiments, the biocompatible solvent comprises an aqueous solution of a therapeutic agent.

Some embodiments are directed to a method of preparing a customizable lyophilized agent, comprising: mixing a biocompatible hydrogel with nanoparticles to form a mixture; freezing the mixture; and lyophilizing the frozen mixture to produce a lyophilized agent, wherein the lyophilized agent is reconstitutable with a biocompatible solvent to form a colloid, and wherein the nanoparticles provide imaging contrast for at least one imaging modality.

In some embodiments, the biocompatible hydrogel comprises chitosan and

sodium alginate.

In some embodiments, the nanoparticles comprise at least one of manganese oxide nanoparticles, cerium oxide nanoparticles, gold nanoparticles, titanium dioxide nanoparticles, or gadodiamide nanoparticles.

In some embodiments, the nanoparticles provide imaging contrast for both computed tomography (CT), magnetic resonance imaging (MRI), or a combination thereof.

In some embodiments, the method further comprises incorporating a therapeutic agent within the mixture prior to freezing.

In some embodiments, the therapeutic agent is an antibody, an immunoadjuvant, a small molecule drug, a nucleic acid, a peptide, or a combination thereof.

In some embodiments, the therapeutic agent is an immunoadjuvant.

In some embodiments, the antibody is an anti-CD40 monoclonal antibody.

In some embodiments, the small molecule drug is caflanone.

In some embodiments, the method further comprises reconstituting the lyophilized agent with an aqueous solution of the therapeutic agent.

In some embodiments, the biocompatible solvent comprises an aqueous solution of a therapeutic agent.

In some embodiments, radiotherapy may be administered to the subject based on imaging data obtained from the at least one imaging modality. The imaging data may provide information about the location and size of the tumor site, as well as the distribution and persistence of the lyophilized agent within the tumor site. This information may be used to guide the delivery of radiotherapy to the tumor site, ensuring that the radiotherapy is accurately and precisely targeted to the tumor while minimizing exposure to neighboring healthy tissues.

In some aspects, the lyophilized agent may offer advantages for storage and handling compared to liquid fiducials. For instance, the lyophilized agent may be stored at higher temperatures than liquid fiducials, reducing the need for cold chain logistics. This may be particularly beneficial in low-resource settings where refrigeration facilities may be limited or unavailable. Furthermore, the lyophilized agent may have reduced volume and weight compared to liquid fiducials, simplifying handling and transportation. This may facilitate the use of the lyophilized agent in various clinical settings, including remote or rural areas where transportation and storage facilities may be limited.

In some aspects, the lyophilized agent may be particularly suitable for use in low-resource settings. The lyophilized form of the agent may offer increased stability compared to liquid fiducials, potentially extending the shelf-life of the product. This increased stability may be beneficial for storage and transport, especially in settings where refrigeration facilities may be limited or unavailable. Furthermore, the lyophilized agent may be stored and transported at higher temperatures than liquid fiducials, reducing the need for cold chain logistics. This may facilitate the use of the lyophilized agent in various clinical settings, including remote or rural areas where transportation and storage facilities may be limited.

In some cases, the lyophilized agent may be compatible with advanced radiotherapy techniques, such as proton therapy, including proton FLASH radiotherapy. The nanoparticles incorporated within the lyophilized matrix may provide imaging contrast for various imaging modalities, including CT and MRI, without producing imaging artifacts. This may allow for the implementation of the lyophilized agent with various radiotherapy techniques, enhancing the precision and accuracy of the radiotherapy treatment.

In some embodiments, the lyophilized agent may be freeze-dried at a temperature of −88° C. and a pressure of 0.12 mBar. This freeze-drying process may involve the removal of water from the frozen mixture of the biocompatible hydrogel and nanoparticles by sublimation and desorption, producing the lyophilized agent. The lyophilized agent may be in the form of a dry, porous matrix that can be easily reconstituted with a biocompatible solvent to form a colloid.

In some aspects, the lyophilized agent may be administered using standard equipment, such as thin needles (≥25 G) and flexible scopes. This may facilitate the administration of the lyophilized agent to various treatment sites in the subject's body, including hard-to-reach tumors or tumors located at or near organs at risk. The administration of the lyophilized agent may involve injecting the colloid into a tumor site or other treatment site in the subject's body.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLES

### Example 1

Studies with Pancreatic Cancer Cells

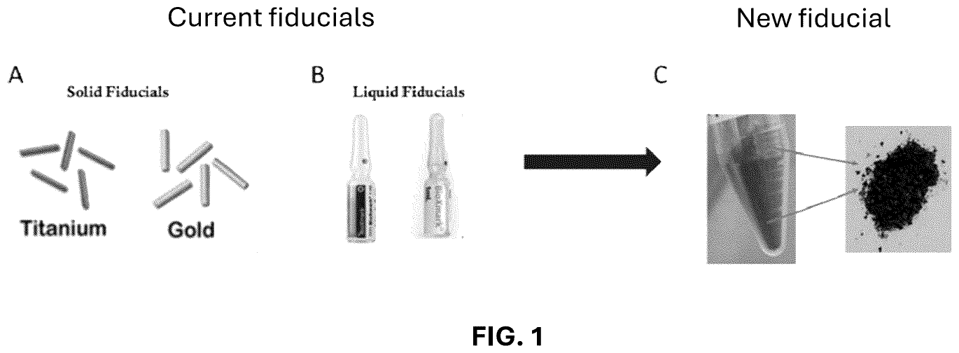

CLARITY Biomaterial Production-2% (w/v) Chitosan hydrogel was prepared in 1% acetic acid in a vial and 4% (w/v) sodium alginate hydrogel in deionized water in a separate vial. The 2 hydrogels were mixed in a 1:1 ratio and mixed with 1½ of either manganese oxide, cerium oxide, or gold nanoparticles to provide imaging contrast throughout radiotherapy treatment. This mixture of the 2 hydrogel and nanoparticles was aliquoted into falcon tubes and allowed to initially freeze at −80 C for at least 24 hours before lyophilization. The frozen samples are lyophilized using a freeze dryer at −88° C. and 0.12 mBar for up to 72 hours. The lyophilized sample from the freeze dryer mimics the shape of the falcon tube container and resembles a meshy dry substance that crumbles upon touch. Therefore, the lyophilized samples are then pulverizedin a mortar and pestle to obtain a powder as illustrated in FIGS. 1 and 2. The lyophilized samples known as CLARITY Biomaterial can be rehydrated using an immunoadjuvant drug solution. Once the CALRITY Biomaterial is rehydrated with the immunoadjuvant solution, it is sonicated for 5 min to obtain a more homogenized solution.

Nanoparticle Tracking Analysis (NTA)—The size distribution of the CLARITY Biomaterial was assessed in solution of the immunoadjuvant drug. The size distribution provides an initial assessment of the range of sizes found when the powder is rehydrated with either anti-CD40 or caflanone. Particle quantification was performed using the ZetaView BASIC NTA-Nanoparticle Tracking Video Microscope PMX-120 (Particle Metrix) in scatter mode with the following capture settings: sensitivity 60-65, shutter 100, and minimum trace length 10. Capture was performed at medium video settings, corresponding to 30 frames per position. ZetaView software version 8.5.10 was used to analyze the recorded videos with the following settings: minimum brightness 30, maximum brightness 255, minimum area 10, and maximum area 1000.

Animal Studies with KPC Syngeneic Pancreatic Cancer Cells-KPC cell line derived from an LS-Kras; p53+/floxed, Pdx-cre mouse was utilized. The pancreatic cancer cell line, KPC, obtained from ATCC was cultured in Dulbecco's Modified Eagle's Medium (DMEM) with 5% Fetal clone II FBS and 1% penicillin/streptomycin. All cells were cultured at 37° C. in a humidified incubator with 5% CO₂. Immunocompetent wild-type C57BL/6 strain female mice were acquired from Charles River at 6 weeks old. They were inoculated 4 weeks following their arrival subcutaneously with 9.4×10⁴ pancreatic cancer cells procured in 100 μL volume of cells per flank. Following cancer cells injection, the tumors were allowed to grow over 2 weeks to at least 3.0 mm or greater in diameter size before the start of treatment.

Pancreatic Tumor Mice CT and MR Imaging-Mice were anesthetized with isoflurane before any computed tomography or magnetic resonance images were taken. In vivo whole-body CT imaging was performed. Mice-bearing pancreatic (KPC) subcutaneous tumors inoculated with CLARITY Biomaterial formulated with Mn₂O₃ and loaded with 800 μg of caflanone were imaged using 50 k Vp x-ray voltage and reconstructed to 0.16 mm³ sized voxels. CT images were visualized with Vivoquant 2020. All MR images were acquired on a multi-nuclear BioSpec 70/30 PET-MR 7T scanner. The mice were anesthetized under ˜1.5-2% isoflurane. First, quick whole-body coronal images were acquired to localize the tumor using a T2-weighted fast spin-echo rapid imaging with refocused echo (RARE) sequence (repetition time, 3 s; echo time, 30 ms; RARE factor, 8; number of averages, 1). T1 weighted axial, coronal, and sagittal images were then acquired with RARE sequence (repetition time, 600 ms; echo time, 7 ms; RARE factor, 2; the number of averages, 6; matrix size, 133×133; field of view, 3.2×3.2 cm; slice thickness, 1 mm; in-plane resolution, 240×240 μm, slice numbers, 20 to 25).

MRI and CT Acquisition for the Human Cadaver—To further assess the feasibility of the CLARITY Biomaterial in providing image guidance, contrast imaging was conducted in a human cadaver. Refrigerated, unfixed cadaveric specimen was implanted in 2 regions: a) their right kidney with CLARITY Biomaterial formulated with manganese oxide nanoparticles; and b) The breast of the human cadaver was inoculated with CLARITY Biomaterial formulated with cerium oxide nanoparticles. CT simulation (TOSHIBA Helical CT scan with 2 mm slice thickness, 120 kVp, and X-ray tube current of 100 mA) was done with the cadaveric specimen in the supine position. The human cadaver specimen was subsequently imaged again. A Philips Achieva 3.0 T MRI System with BODY Transmit Coil was used for MRI acquisition with a repetition time of 5.31 ms, a flip angle of 100, a percent phase field of view of 70.833, and a slice thickness of 0.9 mm. CT and MR imaging were done before and after injection of the CLARITY Biomaterial formulations.

Electron Microscopy Analysis—Transmission Electron Microscopy (TEM): Negative Stain. Samples (8 μL) were adsorbed to glow discharged (EMS GloQube) ultra-thin (UL) carbon-coated 400 mesh copper grids (EMS CF400-Cu-UL), by floatation for 2 min. Grids were rinsed in 3 drops (5 sec each) of TBS (Tris-buffered saline) and negatively stained in 2 consecutive drops of 1% uranyl acetate (UA, aq.), and quickly aspirated. Grids were im-aged on a Hitachi 7600 TEM operating at 80 kV with an AMT XR80 CCD (8 megapixels).

Nanoparticles used for Image-guidance—In addition to manganese oxide (Mn₂O₃), Cerium Oxide (CeO₂), and Gold (GNPs) nanoparticles were used to provide imaging contrast in either mice subcutaneous tumors or a human cadaveric specimen.

Statistical Analysis-Group pairs were compared using a two-tailed Student's t-test. All reported tests were two-tailed and were considered significant at (ns, means not substantial, *p<0.05; *** p<0.001). Survival assays were plotted using GraphPad Prism and were analyzed using Log-rank (Mantel-Cox) and Gehan-Breslow Wilcoxon tests. Error bars are SD unless otherwise noted.

Characterization of CLARITY Biomaterial-Illustrations of the CLARITY Biomaterial in its natural powder form formulated with either gold nanoparticles (GNP) as shown in FIG. 3A or manganese oxide nanoparticles (Mn₂O₃) as displayed in FIG. 3B. Transmission electron microscopy images are shown in FIGS. 3-6 highlighting the size and micromorphology of the CLARITY Biomaterial formulated with manganese oxide (Mn₂O₃) nanoparticles (FIG. 4A and FIGS. 14-16) or loaded with either anti-CD40 monoclonal antibody (FIG. 4B and FIGS. 14-16) or caflanone (FIG. 4C and FIGS. 14-16). Nanoparticle tracking analysis of the CLARITY Biomaterial Mn₂O₃ reconstituted with either caflanone or anti-CD40 showed a clear distinction of its size distribution in solution (size range=100-300 nm) for the caflanone (FIGS. 5A-B) versus the anti-CD40 (size range=63-210 nm) (FIGS. 6A-B) loaded CLARITY Biomaterial respectively.

Image guidance of CALRITY Biomaterial in Pancreatic Cancer—This study focused on investigating a lyophilized powder that can be customized using different immunoadjuvants to provide image guidance and also boosting therapeutic outcome in pre-clinical mouse models of pancreatic cancer. CLARITY biomaterial was evaluated for providing both CT and MRI contrast over time when administered in the pancreatic tumor tissue. FIGS. 7-8 highlight the image guided capability of the CLARITY Biomaterial in providing MRI contrast for up to 30-days post treatment. FIGS. 9-10 display the CT contrast provided by the CLARITY Biomaterial for up to 30-days where the contrast is no longer visible.

Manganese oxide offers significant multi-functionality in the CLARITY Biomaterial to provide both CT and MRI contrast over time during radiotherapy. FIGS. 7-10 pinpoints that the contrast provided by the manganese oxide nanoparticles is brighter in MR images and last beyond the 30-days of observation compared to the CT contrast provided by these same nanoparticles. However, FIGS. 17-18 showed CT contrast for up to 30-days post-treatment. Human cadaveric specimens were used to further evaluate the CT and MRI contrast provided by the CLARITY Biomaterial formulated with Mn₂O₃ as shown in FIG. 11 in the right kidney of the specimen as indicated by the yellow arrow post-injection. CLARITY Biomaterial formulated with cerium oxide interestingly also showed both CT and MRI contrast in the breast tissue of the specimen in FIG. 12. An additional functionality of the manganese oxide nanoparticles is the potential to address hypoxia, which is critical for enhancing radiotherapy and overcoming immunosuppression.

Efficacy of CLARITY Biomaterial in Pancreatic Cancer-CLARITY Biomaterial was evaluated for its therapeutic capability when loaded with either 100 μg anti-CD40 monoclonal antibody or 800 μg caflanone drug against a no-treatment group for C57BL6 mice bearing subcutaneous pancreatic tumors. Significant delay in tumor growth was observed for the group that received CLARITY Biomaterial Mn₂O₃ loaded with 100 μg of anti-CD40 (*, p=0.0212) compared to no treatment group (FIG. 13A). In addition, significant mice survival was also observed for the cohorts treated with either CLARITY Biomaterial loaded with 100 ug of Anti-CD40 (***, p=0.0006) or CLARITY Biomaterial loaded with 800 μg of FBL-03G (*, p=0.0124) from FIG. 13B compared to the no treatment group.

### Example 2

### Materials and Methods

### Materials

Dimethyl sulfoxide (DMSO), Sodium Alginate and chitosan were acquired from Sigma-Aldrich (St. Louis, MO, USA)). All cell culture products (DMEM, Trypsin, Fetal Bovine Serum, penicillin/streptomycin, and PBS pH 7.4) were found from ThermoFisher, and Life Technologies (Waltham, MA, USA). Flavocure Biotech Inc. (Baltimore, MD, USA) supplied the test molecule, Caflanone (FBL-03G) with a purity of 98.7% determined by High Performance Liquid Chromatography (HPLC). Manganese Oxide (Mn2O3) Nanopowder/Nanoparticles Water Dispersion (20 wt %, APS: 30 nm, Purity: 99.2%, Stock #: US3340, Mn2O3 Nanoparticles SSA: 150 m²/g) were obtained from U.S. Nanomaterials Inc. (Houston, TX, USA). A lyophilizer, FreeZone 4.5 PLU Liter Benchtop Freeze Dry System from Labconco Corporation (Kansas City, MO, USA) was utilized to render the colloid samples into the CLARITY biomaterial powder as emphasized in previous work.

### Clonogenic Survival Assay

Actively growing monolayers of KPC, MIA PaCa-2 cancer cells and L929 fibroblast 104 cells were trypsinized and 500 to 1000 cells per well respectively were seeded in 10-cm 105 dishes for KPC and MIA-PaCa2 and 6 well plates for L929. 24 hours later, seeded cancer 106 cells were treated with 10, 50, 100, 200, 300, 400, 500 or 1000 UM of Caflanone (FBL-03G). 107 L929 was also treated 24 hours later with 1, 3.5, 10 μM of Caflanone. The cancer cells were 108 irradiated at 0, 2, 4, 6, 8 or 10 Gy using 220 k Vp energy, 13 mA, 24 h after drug treatment. 109 A machine called CIXD by Xstrahl was used to deliver external beam radiation. The growing colonies (≥50 cells/colony) were fixed with 4% formalin and stained with 0.5% (w/v) of crystal violet 9-12 days after treatment. Colonies were counted automatically using Interscience Scan 4000 colony counter and a percent survival was calculated following standard protocol.

### Data Collection and Imaging

The animals were scanned using an MRS*DRYMAG 7024 7.0T MRI system (MR Solutions, UK) located at the Center for Infection and Inflammation Imaging Research (Ci3R), Johns Hopkins University School of Medicine. Mice were anesthetized with ˜1.5-2% isoflurane and respiratory rate was continuously monitored throughout the scan using an ERT control/gating module (SA Instruments, Inc. NY, USA) to regulate anesthesia and enable respiratory-gated acquisition. Animals were positioned in a Quadrature mouse whole-body RF coil (MR Solutions, UK). Initially, quick whole-body coronal images were acquired to localize the tumor using a T2-weighted fast spin-echo (T2w-FSE) sequence. Subsequently, T1-weighted (T1w-FSE) images were acquired in the axial, coronal, and sagittal planes using the following parameters: repetition time (TR)=1000 ms; echo time (TE)=11 ms; flip angle=90°; echo train length=4; bandwidth=66.6 kHz; number of averages=2; field of view (FOV)=3.5×3.5 cm; matrix size=256× 248; slice thickness=1 mm; number of slices=24-26; fat saturation applied.

### Cell Culture

A human pancreatic cancer cell line, MIA PaCa-2 cells, was obtained from Dr. Fred Bunz at the Johns Hopkins University Baltimore. KPC cell line was derived from an LS-Kras; p53+/floxed, Pdx-cre mouse. Pancreatic cancer cell line, KPC, obtained from Cancer Research UK (Glasgow Beatson Institute). L929 cell line is a fibroblast like cell line from a C3H/An mouse connective tissue. All cell lines were cultured respectively in Dulbecco's Modified Eagle's Medium (DMEM) with 5% Fetal clone II FBS and 1% penicillin/streptomycin. All cells were cultured at 37° C. in a humidified incubator with 5% CO2.

### Generating Subcutaneous Pancreatic Tumors

C57BL/6 mice were inoculated with 1.5×10⁵ KPC cells per mouse flank on either left or right flank to generate subcutaneous tumors. Once subcutaneous tumors reached palpable size ˜3.0 mm in diameter, the animals were randomized into different cohorts such as No treatment (n=11, 8 males 3 females), Caflanone (n=10, 6 males 4 females), FBL-GS 706 (n=10, 6 males and 4 females), and CLARITY Biomaterial_Caflanone (n=10, 6 males and 4 females). Samples sizes were based on the availability of mice housed in our animal facility. Tumor volume and mice survival were monitored over time. Animal experiments followed the guidelines and regulations set by the Johns Hopkins University Institutional Animal Care and Use Committee (ACUC) with protocol #MO24M298. Mice maintenance in Johns Hopkins University animal facility was in accordance with the Institutional Animal Care and Use Committee approved guidelines. Directly after treatment, a digital Vernier caliper was used to measure the length and width of the subcutaneous tumors. Tumor volume formula used: (length×width²)/2. Measurement along the imaginary longitude to the leg was selected as length and the vertical was for width. The tumors were restrained between the skin surface layers. The tumor volume was plotted against time. Animal survival was performed for treatments following ACUC approved protocol, which was predetermined based on published evidence justifying such a study design. Tumor attainment >=2 cm in diameter on flank, concave ulceration and tumor burst were determined as excessive tumor burden and mouse was euthanized following the approved mice protocol.

### Flow Cytometry

Mouse tumors were harvested and minced into <1 um portions and digested in 5 mg/mL of Collagenase A for 45 min in 37 C water bath and vortex every 10 min during the digestion period. The tumor cells were strained twice using 70 um strainer followed by centrifugation. The collected cells were rinsed twice in cell staining buffer before resuspending the cells in Phosphate Buffered Saline (PBS, pH 7.4). 1×106 cells/sample were stained with Zombie Aqua (BioLegend) as viability dye for 30 min, followed by staining in the presence of Fc block (CD16/32, BioLegend), and murine monoclonal antibodies against CD4 (GK1.5) and CD8a (53-6.7), all from BioLegend. Antibodies were diluted 1:100 in FACS buffer (PBS) and cells were stained at 4 C in the dark for 20 min before acquisition using a CytoFLEX Flow Cytometer (Beckman Coulter), and CytExpert software (Beckman Coulter. Data was analyzed using FlowJo software (version 10.8).

### Electron Microscopy Images

Transmission Electron Microscopy: Samples (8 uL) were adsorbed to glow discharged (EMS GloQube) ultra-thin (UL) carbon coated 400 mesh copper grids (EMS CF400-Cu-UL), by floatation for 2 min. Grids were rinsed in 3 drops (5 sec each) of TBS (Tris-buffered saline) and negatively stained in 2 consecutive drops of 1% uranyl acetate (UA, aq.), and quickly aspirated. Grids were imaged on a Hitachi 7600 TEM operating at 80 kV with an AMT XR80 CCD (8 megapixel). Scanning Electron Microscopy: Samples were mounted on carbon coated stubsand imaged on a ThermoFisher Helios FIB-SEM* or JSM IT700HR In-Touch Scope. *FIB-SEM and JSM IT700HR at Homewood: Material Characterization and Processing Center of JHU and the Hopkins Extreme Materials Institute

### Statistical Analysis

GraphPad prism v9 was used to generate Kaplan-Meier curves as well as all clonogenic assay plots. Additionally, was used to evaluate statistical significance.

### Example 3

### Effect of Flavinoid Drugs on L929, KPC and MIA PaCa2 Cells Proliferation Over Time.

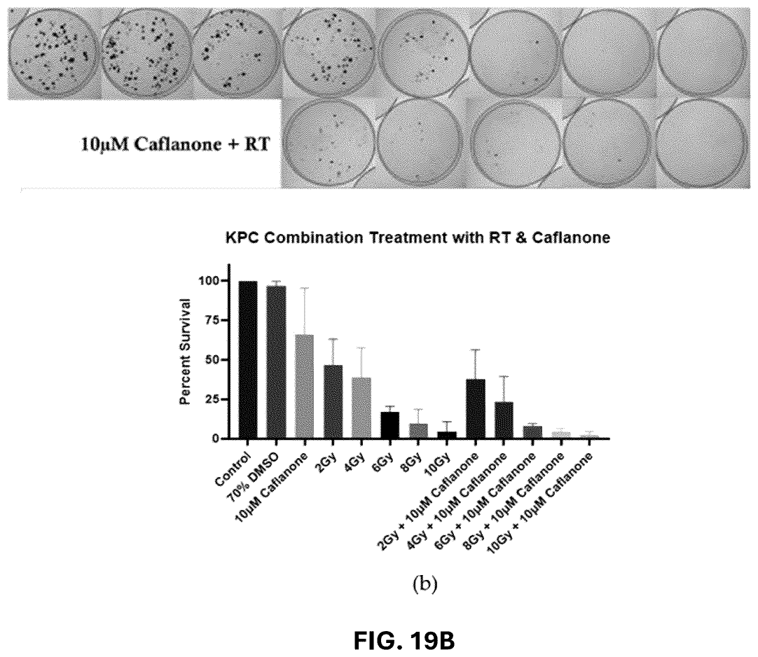

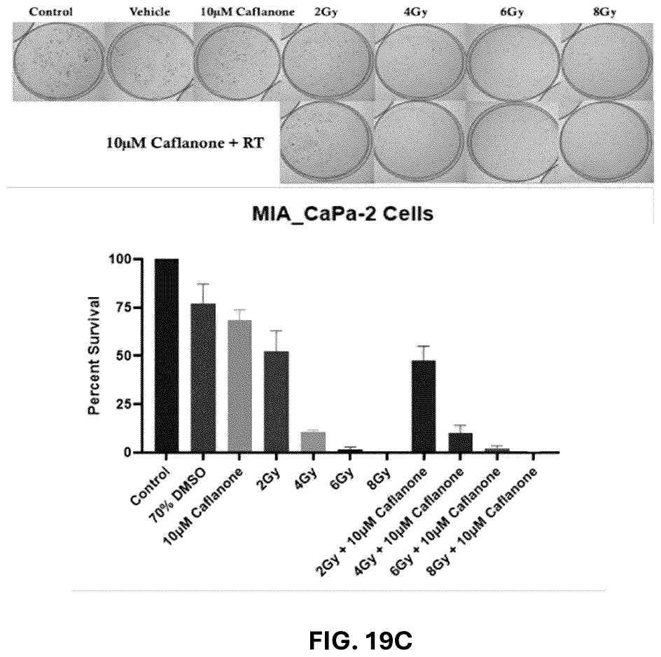

Caflanone and CLARITY Biomaterial were first tested against murine fibroblast L929 cells to determine normal cell toxicity. Concentrations of 1, 3.5, and 10 μM Caflanone were tested and determined the least amount of toxicity (78.6% cell survival) using 10 μM Caflanone which was then further used in the current studies. Additionally, toxicity of CLARITY Biomaterial on the L929 cells was evaluated and determined minimal toxicity (FIGS. 19A-19C). KPC was subsequently treated with 10 μM Caflanone, Radiation alone and Radiation with Caflanone. When adjuvant radiation with Caflanone results demonstrate increased cell kill at each radiation dose than radiation alone as shown in the Table1 below.

TABLE 1
Percent Survival Comparison of KPC cells when treating
Radiation Alone or Radiation with Caflanone

| Dose (Gy) | Radiation | RT + 10 μM |
|---|---|---|
| 2 | 46.6 (%) | 37.7 (%) |
| 4 | 38.8 (%) | 23.1 (%) |
| 6 | 16.8 (%) | 8.1 (%) |
| 8 | 9.7 (%) | 4.6 (%) |
| 10 | 4.4 (%) | 2.5 (%) |

While treating human Pancreatic cancer cell line MIA PaCa2 with 10 μM Caflanone. The greatest change in decreasing cell survival was seen at 2Gy where 2Gy alone had a cell survival at 52.6% but when adjuvanted with Caflanone the cell survival was 47.6%.

### Example 4

### Characterization and T1 Weighted MRI Images of CLARITY Biomaterial

CLARITY biomaterial was evaluated using Transmission Electron microscopy and Scanning Electron microscopy. Transmission Electron microscopy (TEM) images provide detail into the mophology of the CLARITY Biomaterial formulated with magnesium oxide. Scanning Electron microscopy provide detail of the CLARITY biomaterial surface. The TEM images show that CLARITY Biomaterial is a sphere less than 100 microns in size that groups together in solution. In the SEM images there are brighter areas, which result from the Mn₂O₃ nanoparticles. Heavier nuclei and compounds deflect the incident electrons stronger resulting in these brighter regions in the image. This is important since CLARITY has the capability to act as a fiducial marker in image guided radiation therapy.

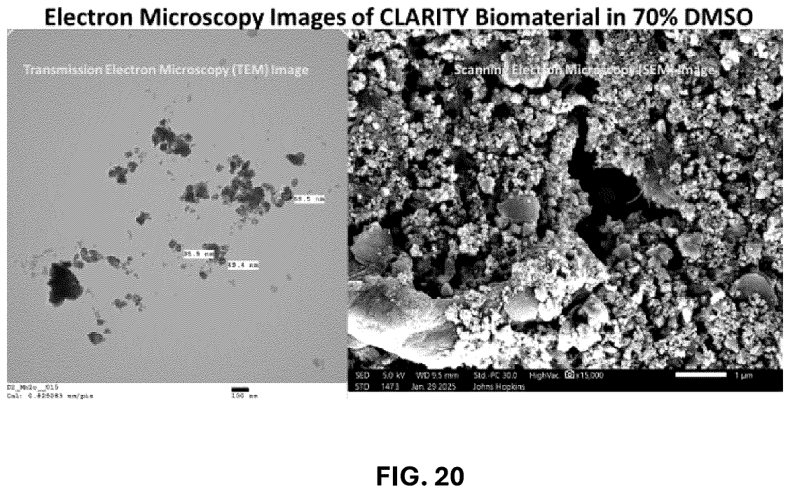

As previously demonstrated CLARITY Biomaterial can be imaged on a CT scan, and to achieve such tumor tracking and image contrast the biomaterial needs to be of higher atomic number than the surrounding tissue. This further demonstrates the ability for CLARITY Biomaterial to be imaged using T1-weighted MRI, Mn₂O₃ exhibits a high paramagnetic moment from the Mn3+ allowing for contrast on the MRI image (FIG. 20).

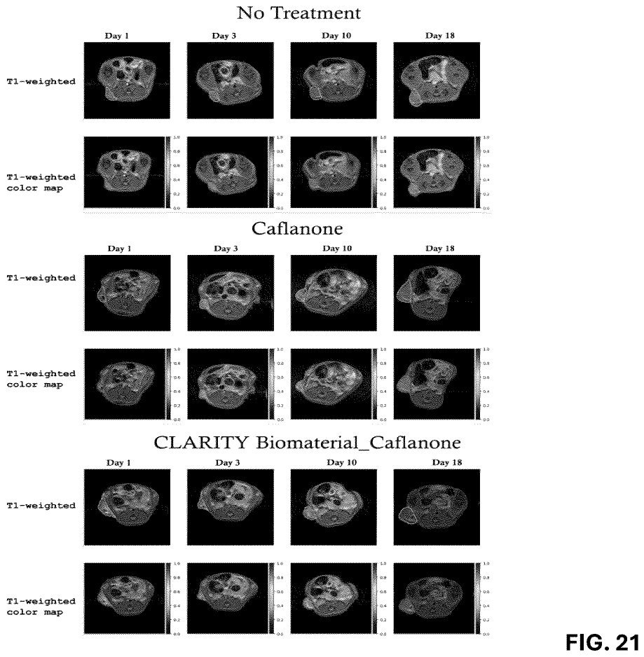

In performing T1 weighed images of mice from the No Treatment, Caflanone and CLARITY Biomaterial loaded with caflanone over time (FIG. 21). CLARITY Biomaterial was visible clearly in T1 weighted image as shown as a bright white in the tumor segmented (yellow outline) region. This was more pronounced in the T1 weighted color map. These results indicate that over the course of treatment, for at least 18 days CLARITY Biomaterial can provide clear image tracking capabilities.

### Example 5

### Efficacy of Single Treatments Compared to CLARITY Biomaterial Loaded Caflanone

CLARITY Biomaterial loaded with Caflanone was evaluated for its ability to control tumor size and enhance survival as opposed to single treatments of Caflanone. CLARITY Biomaterial loaded with caflanone demonstrated greater tumor control in Female bearing mice as well as increased survival in both the males and females. Additionally, immune cell populations of CD4 and CD8 were tested at 23 days post treatment and 43 days post treatment. At both time points there was an increase immune cell population in CLARITY biomaterial loaded with caflanone than single treatments.

In assessing tumor control results demonstrate increased control when treating the tumors with Caflanone or CLARITY Biomaterial_Caflanone in both male and female mice. This was particularly emphasized when treating the female mice with CLARITY Biomaterial_Caflanone where it was seen nearly 5× greater tumor control. Finally, the immune response was assessed the immune response after treating with the flavonoid drugs or CLARITY Biomaterial loaded with Caflanone. On day 23 an increased immune response across all groups was demonstrated in comparison to the no treatment group for both CD4 and CD8. However, on day 43 the results are less coherent, indicating the immune cells may have returned to baseline.

### DISCUSSION

This study focused on the use of flavonoid based drugs in the treatment of pancreatic cancer as well as understanding the difference between single drug treatments and CLARITY biomaterial loaded with drug. Decreased cancer cell survival was shown when treating pancreatic cancer cells with the flavonoid-based drug, Caflanone, and radiation, over standalone drug or radiation. Further in the KPC cell lines that there was increased cancer cell kill when combining radiation with the flavonoid based drug at across all doses of radiation and then in MIA-PaCa2 there was increase cell kill when adjuvating radiation with caflanone at 2 and 4Gy. From a clinical perspective this is important because conventional radiation schemes deliver fractions at 2Gy, and stereotactic body radiation therapy (SBRT) deliver fractions at around 8Gy. Since radiation is delivered over the course of many fractions the repeated use of the immunoadjuvant can accumulate potentially offset any immunosuppression from radiation alone as well as increasing treatment efficacy.

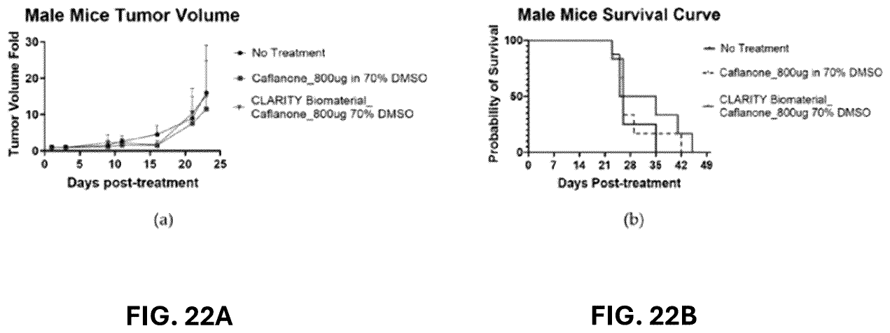

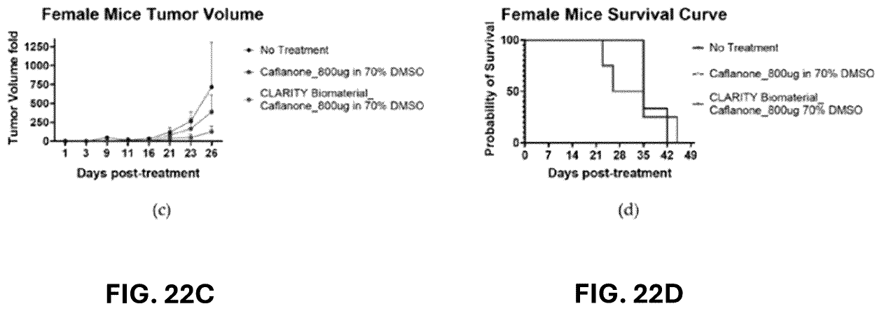

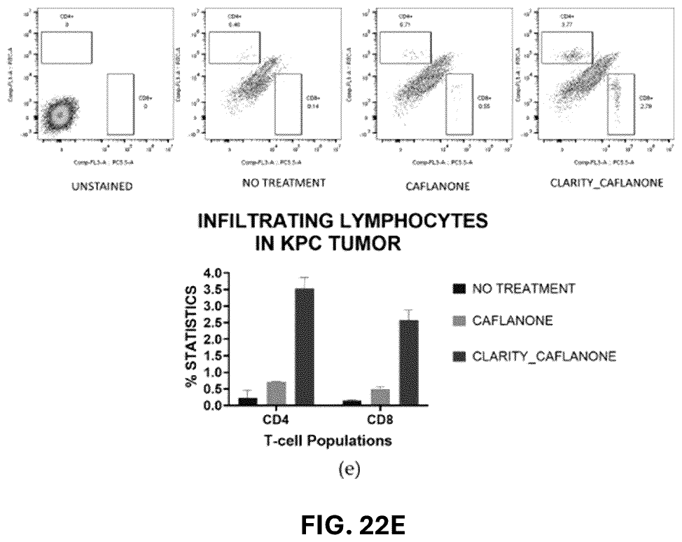

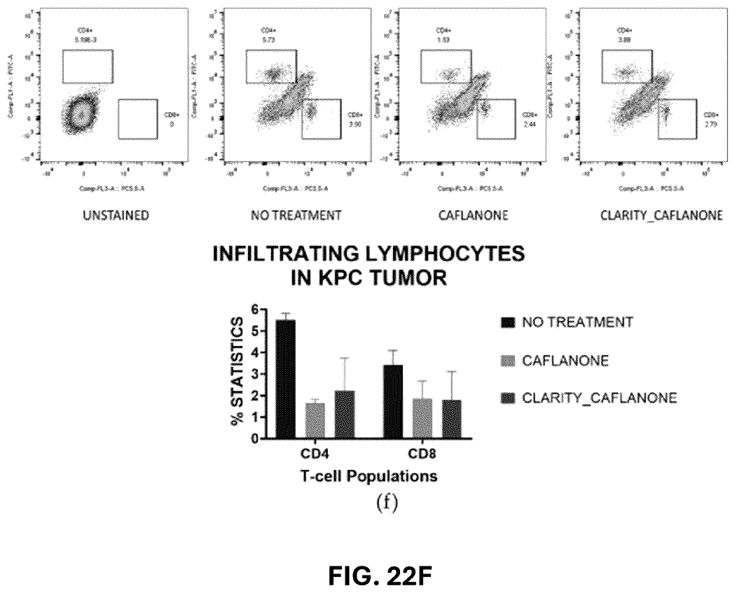

Additionally, there was enhanced survival and immune response when treating mice with im-munoadjuvants. Higher CD4 immune cells were demonstrated in all treatment groups 24 days post treatment in comparison to the control. Additionally, higher CD8+ immune cells in the CLARITY biomaterial loaded with Caflanone than the control as shown in FIGS. 22A-22F. These results demonstrate the potential to use CLARITY Biomaterial in more immunotherapeu-tic applications.

### CONCLUSIONS

This study investigated the efficacy of flavonoid based drugs as an immunoadjuvant. Improved cell kill while using the immunoadjuvants in combination with radiation therapy indicating further use of this combination in immune cold tumors was demonstrated. Additionally, there was an enhanced immune response and lower toxicity when using smart biomaterials like CLARITY loaded with these immune drugs. Finally, there was 5× greater tumor control when loading caflanone into CLARITY Biomaterial. These results further use of smart radiotherapy biomaterials that can be loaded with these immunoadjuvants to improve radiation therapy and reduce toxicities.

Although the invention has been described with reference to the above examples, it will be understood that modifications and variations are encompassed within the spirit and scope of the invention. Accordingly, the invention is limited only by the following claims.

1. A customizable lyophilized agent, comprising:
a biocompatible lyophilized matrix; and
nanoparticles incorporated within the lyophilized matrix,
wherein the lyophilized matrix is reconstitutable with a biocompatible solvent to form a colloid, and wherein the nanoparticles provide imaging contrast for at least one imaging modality.

2. The customizable lyophilized agent of claim 1, wherein the nanoparticles comprise at least one of manganese oxide nanoparticles, cerium oxide nanoparticles, gold nanoparticles, titanium dioxide nanoparticles, or gadodiamide nanoparticles.

3. The customizable lyophilized agent of claim 2, wherein the nanoparticles provide imaging contrast for computed tomography (CT), magnetic resonance imaging (MRI), or a combination thereof.

4. The customizable lyophilized agent of claim 1, wherein the biocompatible lyophilized matrix comprises chitosan and sodium alginate.

5. The customizable lyophilized agent of claim 1, further comprising a therapeutic agent incorporated within the lyophilized matrix.

6. The customizable lyophilized agent of claim 5, wherein the therapeutic agent is an antibody, an immunoadjuvant, a small molecule drug, a nucleic acid, a peptide, or a combination thereof.

7. The customizable lyophilized agent of claim 6, wherein the therapeutic agent is an immunoadjuvant.

8. The customizable lyophilized agent of claim 6, wherein the antibody is an anti-CD40 monoclonal antibody and wherein the small molecule drug is caflanone.

9. (canceled)

10. The customizable lyophilized agent of claim 5, wherein the lyophilized matrix is reconstitutable with an aqueous solution of the therapeutic agent.

11. A method of imaging a subject using a customizable lyophilized agent, comprising:
reconstituting a lyophilized agent with a biocompatible solvent to form a colloid, wherein the lyophilized agent comprises a biocompatible lyophilized matrix and nanoparticles incorporated within the lyophilized matrix;
administering the colloid to the subject; and
imaging the subject using at least one imaging modality, wherein the nanoparticles provide imaging contrast for the at least one imaging modality.

12. The method of claim 11, wherein the at least one imaging modality comprises computed tomography (CT), magnetic resonance imaging (MRI), or a combination thereof.

13. (canceled)

14. The method of claim 11, further comprising administering radiotherapy to the subject based on imaging data obtained from the at least one imaging modality.

15. The method of claim 11, wherein the biocompatible solvent comprises an aqueous solution of a therapeutic agent.

16-19. (canceled)

20. A method of treatment using image-guided radiation therapy (IGRT) comprising administering to the subject the customizable lyophilized agent of claim 1.

21. The method of claim 20, wherein the treatment is for cancer.

22. The method of claim 21, wherein the cancer is arteriovenous malformations, bone cancer, brain cancer, breast cancer, chondrosarcoma, chordoma, colon cancer, esophageal cancer, Ewing's sarcoma, head and neck cancers, leukemia, liver cancer, lung cancer, lymphoma, metastatic brain cancer, meningioma, neuroma, non-Hodgkin's lymphoma, osteosarcoma, pancreatic cancer, paranasal sinus cancer, prostate cancer, soft tissue sarcomas, spinal cord tumors, trigeminal neuralgia, or a combination thereof.

23. The method of claim 22, wherein the cancer is pancreatic cancer.

24. The method of claim 20, further comprising administering a therapeutic agent.

25-29. (canceled)

30. A method of preparing a customizable lyophilized agent, comprising:
mixing a biocompatible hydrogel with nanoparticles to form a mixture;
freezing the mixture; and
lyophilizing the frozen mixture to produce a lyophilized agent,
wherein the lyophilized agent is reconstitutable with a biocompatible solvent to form a colloid, and wherein the nanoparticles provide imaging contrast for at least one imaging modality.

31-34. (canceled)

35. The method of claim 30, further comprising incorporating a therapeutic agent within the mixture prior to freezing.

36-41. (canceled)
